(19) United States
(12) Patent Application Publication
(10) Pub. No.: US 2019/0045170 A1
(43) Pub. Date: Feb. 7, 2019

(54) MEDICAL IMAGE PROCESSING DEVICE, SYSTEM, METHOD, AND PROGRAM

(71) Applicant: SONY CORPORATION, Tokyo (JP)

(72) Inventors: Yuki SUGIE, Kanagawa (JP); Takeshi UEMORI, Tokyo (JP); Kenji TAKAHASI, Kanagawa (JP); Tsuneo HAYASHI, Tokyo (JP)

(73) Assignee: SONY CORPORATION, Tokyo (JP)

(21) Appl. No.: 16/073,911

(22) PCT Filed: Jan. 10, 2017
(86) PCT No.: PCT/JP2017/000425

§ 371 Date: Jul. 30, 2018

(30) Foreign Application Priority Data
Feb. 24, 2016 (JP) 2016-033034

(51) Int. Cl. H04N 13/128; G06T 7/00; G06T 7/11; G06T 7/174; G06T 3/40; A61B 90/00

(52) U.S. Cl. CPC H04N 13/128; G06T 7/0012; G06T 7/11; G06T 7/174; A61B 1/00193; A61B 90/37; G06T 2207/20228; G06T 2207/10048; G06T 2207/10012; G06T 3/40

(57) ABSTRACT

[Object] To improve accuracy of determination of a disparity for medical image processing.

[Solution] There is provided a medical image processing device including: a region determination unit configured to determine a non-living body region in a visual field by using a region-determination image showing the visual field that is same as a visual field that a disparity-determination image shows; and a disparity determination unit configured to determine at least a disparity to be applied to the non-living body region by using a result of the determination of the non-living body region performed by the region determination unit and the disparity-determination image.

### TECHNICAL FIELD

The present disclosure relates to a medical image processing device, system, method, and program.

### BACKGROUND ART

Conventionally, there has been known a technology of using a plurality of images generated by capturing an image of a subject from different points of sight to determine a disparity between the points of sight for generating a stereoscopic image or another use (e.g., see Patent Literature 1 and Patent Literature 2). For example, according to a block matching method using two images from a stereo camera, a position of a certain block in one image including a small image having the highest similarity to a small image of a block in the other image is searched for, and a disparity is determined on the basis of a difference between the positions of the blocks obtained as a result of the search.

### CITATION LIST

### Patent Literature

Patent Literature 1: JP 2014-206893A

Patent Literature 2: JP 2015-146526A

### DISCLOSURE OF INVENTION

### Technical Problem

However, in the field of medical image processing, in some cases, high visibility is demanded in a case of, for example, minute subjects such as a needle tool and a thread for use in suturing or ligation in surgical operation, and the conventional block matching method cannot sufficiently satisfy such a demand.

### Solution to Problem

According to the present disclosure, there is provided a medical image processing device including: a region determination unit configured to determine a non-living body region in a visual field by using a region-determination image showing the visual field that is same as a visual field that a disparity-determination image shows; and a disparity determination unit configured to determine at least a disparity to be applied to the non-living body region by using a result of the determination of the non-living body region performed by the region determination unit and the disparity-determination image.

In addition, according to the present disclosure, there is provided a medical image processing system including: the medical image processing device; and an imaging device configured to capture an image of a subject in the visual field and generate at least one of the disparity-determination image and the region-determination image.

In addition, according to the present disclosure, there is provided a medical image processing method including: determining a non-living body region in a visual field by using a region-determination image showing the visual field that is same as a visual field that a disparity-determination image shows; and determining at least a disparity to be applied to the non-living body region by using a result of the determination of the non-living body region and the disparity-determination image.

In addition, according to the present disclosure, there is provided a program for causing a processor that controls a medical image processing device to function as: a region determination unit configured to determine a non-living body region in a visual field by using a region-determination image showing the visual field that is same as a visual field that a disparity-determination image shows; and a disparity determination unit configured to determine at least a disparity to be applied to the non-living body region by using a result of the determination of the non-living body region performed by the region determination unit and the disparity-determination image.

### Advantageous Effects of Invention

According to the technology of the present disclosure, it is possible to improve accuracy of determination of a disparity for medical image processing and achieve high visibility of a subject.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is an explanatory diagram for describing a schematic configuration of a medical image processing system according to an embodiment.

FIG. 2 is an explanatory view illustrating an example of a disparity-determination image showing a minute subject.

FIG. 3 is an explanatory diagram for describing accuracy of a disparity determined in accordance with an existing block matching method.

FIG. 4 is a block diagram illustrating an example of a configuration of an imaging device according to a first embodiment.

FIG. 5 is a block diagram showing an example of a configuration of an image processing device according to a first embodiment.

FIG. 6 is an explanatory diagram for describing spectral characteristics of several materials.

FIG. 7 is an explanatory diagram for describing an example of analysis of spectral characteristics of subjects.

FIG. 8 is an explanatory diagram for describing an example of switching of wavelength patterns of irradiation light.

FIG. 9A is an explanatory diagram for describing determination of a disparity using a compound-eye stereo camera.

FIG. 9B is an explanatory diagram for describing determination of a disparity using a monocular stereo camera.

FIG. 10 is an explanatory diagram for describing an example of a result of determination of a non-living body region using a region-determination image.

FIG. 11 is an explanatory diagram for describing an example of a collation block that is set on the basis of a result of determination of a non-living body region.

FIG. 12A is an explanatory diagram for describing a first example of setting a weight to a collation block.

FIG. 12B is an explanatory diagram for describing a second example of setting a weight to a collation block.

FIG. 13 is an explanatory diagram for describing an example of setting a plurality of collation blocks.

FIG. 14 is a flowchart showing an example of the whole flow of image processing according to the first embodiment.

FIG. 15 is a flowchart showing an example of a detailed flow of region-determination image acquisition processing shown in FIG. 14.

FIG. 16 is a flowchart showing an example of a detailed flow of region determination processing shown in FIG. 14.

FIG. 17A is a flowchart showing a first example of a detailed flow of collation block setting processing shown in FIG. 14.

FIG. 17B is a flowchart showing a second example of a detailed flow of collation block setting processing shown in FIG. 14.

FIG. 18 is a flowchart showing an example of a detailed flow of disparity determination processing shown in FIG. 14.

FIG. 19 is a block diagram illustrating an example of a configuration of an imaging device according to a second embodiment.

FIG. 20 is a block diagram illustrating an example of a configuration of an image processing device according to a second embodiment.

FIG. 21 is an explanatory diagram for describing another example of a result of determination of a non-living body region using a region-determination image.

FIG. 22 is a flowchart showing an example of the whole flow of image processing according to the second embodiment.

FIG. 23 is a flowchart showing an example of a detailed flow of region determination processing shown in FIG. 22.

FIG. 24 is a block diagram illustrating an example of a configuration of an imaging device according to a third embodiment.

FIG. 25 is a block diagram illustrating an example of a configuration of an image processing device according to a third embodiment.

FIG. 26 is an explanatory diagram for describing further another example of a result of determination of a non-living body region using a region-determination image.

FIG. 27 is a flowchart showing an example of the whole flow of image processing according to the third embodiment.

FIG. 28 is a flowchart showing an example of a detailed flow of region determination processing shown in FIG. 27.

### MODE(S) FOR CARRYING OUT THE INVENTION

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

Further, description will be provided in the following order.

1. Introduction
1-1. Overview of system
1-2. Description of problem

2. First embodiment
2-1. Configuration example of imaging device
2-2. Configuration example of image processing device
2-3. Flow of processing

3. Second embodiment
3-1. Configuration example of imaging device
3-2. Configuration example of image processing device
3-3. Flow of processing

4. Third embodiment
4-1. Configuration example of imaging device
4-2. Configuration example of image processing device
4-3. Flow of processing

5. Conclusion

### 1. Introduction

### [1-1. Overview of System]

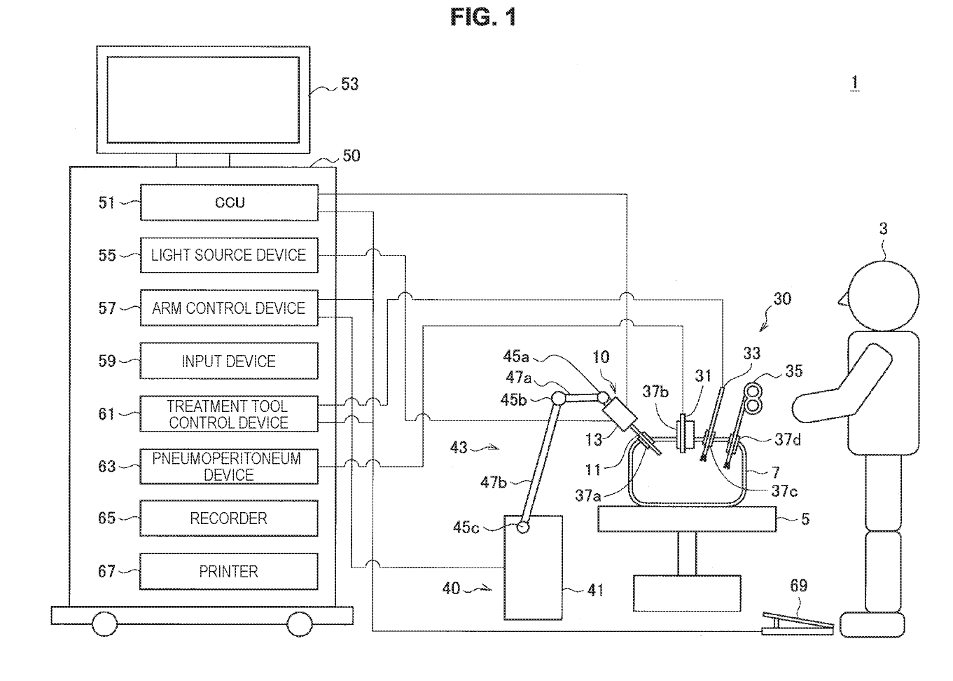

In this section, an overview of an example system to which a technology according to the present disclosure is applicable will be described. FIG. 1 illustrates an example of a schematic configuration of a medical image processing system 1 according to an embodiment. The medical image processing system 1 is an endoscopic surgery system. In the example of FIG. 1, a practitioner (doctor) 3 performs endoscopic surgery by using the medical image processing system 1 on a patient 7 on a patient bed 5. The medical image processing system 1 includes an endoscope 10, other surgical instruments (operation instruments) 30, a support arm device 40 that supports the endoscope 10, and a cart 50 on which various devices for endoscopic surgery are mounted.

In endoscopic surgery, an abdominal wall is punctured with a plurality of cylindrical opening tools 37a to 37d called trocars, instead of being cut to open an abdomen. Then, a lens barrel 11 of the endoscope 10 and the other operation instruments 30 are inserted into a body cavity of the patient 7 through the trocars 37a to 37d. In the example of FIG. 1, a pneumoperitoneum tube 31, an energy treatment device 33, and a forceps 35 are illustrated as the other operation instruments 30. The energy treatment device 33 is used for treatment such as incision or separation of tissue or sealing of a blood vessel with a high-frequency current or ultrasonic vibration. Note that the illustrated operation instruments 30 are merely examples, and other types of operation instruments (e.g., thumb forceps, retractor, or the like) may be used.

An image of the inside of the body cavity of the patient 7 captured by the endoscope 10 is displayed by a display device 53. The practitioner 3 performs, for example, treatment such as excision of an affected part by using the energy treatment device 33 and the forceps 35 while viewing the display image in real time. Note that, although not illustrated, the pneumoperitoneum tube 31, the energy treatment device 33, and the forceps 35 are supported by a user such as the practitioner 3 or an assistant during surgery.

The support arm device 40 includes an arm portion 43 extending from a base portion 41. In the example of FIG. 1, the arm portion 43 includes joint portions 45a, 45b, and 45c and links 47a and 47b and supports the endoscope 10. As a result of driving the arm portion 43 under the control of an arm control device 57, a position and posture of the endoscope 10 can be controlled, and fixation of a stable position of the endoscope 10 can also be achieved.

The endoscope 10 includes the lens barrel 11 and a camera head 13 connected to a base end of the lens barrel 11. Part of the lens barrel 11, which has a certain length from a tip thereof, is inserted into the body cavity of the patient 7. In the example of FIG. 1, the endoscope 10 is configured as a so-called rigid endoscope having a rigid lens barrel 11. However, the endoscope 10 may be configured as a so-called flexible endoscope.

An opening into which an objective lens is fit is provided at the tip of the lens barrel 11. A light source device 55 is connected to the endoscope 10, and light generated by the light source device 55 is guided to the tip of the lens barrel by a light guide extended in the lens barrel 11, and an observation target in the body cavity of the patient 7 is irradiated with the light via the objective lens. Note that the endoscope 10 may be a forward-viewing endoscope, a forward-oblique viewing endoscope, or a lateral-viewing endoscope.

The camera head 13 includes an irradiation unit, an optical system, a drive system, and an imaging unit including an image sensor. The irradiation unit irradiates a subject with irradiation light supplied from the light source device 55 via the light guide. The optical system typically includes a lens unit and collects observation light (reflected light of irradiation light) from a subject, the observation light being taken in through the tip of the lens barrel 11, toward the image sensor. Positions of a zoom lens and a focus lens in the lens unit are changeable by being driven by the drive system in order to variably control imaging conditions such as a magnification and a focal distance. The image sensor of the camera head 13 performs photoelectric conversion on the observation light collected by the optical system and generates an image signal serving as an electric signal. The image sensor may be a 3CCD sensor including individual imaging elements that generate image signals of respective three color components or may be another type of image sensor such as a 1CCD image sensor or a 2CCD image sensor. The image sensor may include, for example, any type of imaging element such as a complementary metal oxide semiconductor (CMOS) or a charge-coupled device (CCD). The image signals generated by the image sensor are transmitted as RAW data to a camera control unit (CCU) 51.

In a certain embodiment, a captured image shown by an image signal generated by the camera head 13 includes a disparity-determination image. The disparity-determination image typically includes a right-eye image and a left-eye image. The right-eye image and the left-eye image may be generated by a right-eye image sensor and a left-eye image sensor of a compound-eye camera, respectively.

Instead of this, the right-eye image and the left-eye image may be generated by a single image sensor of a monocular camera (e.g., by a shutter switching method). Further, the camera head 13 can also generate an image signal of a region-determination image to be used for determining a non-living body region in a visual field. The region-determination image may be a visible light image in a certain embodiment, and the region-determination image may be an infrared image in another embodiment.

The CCU 51 is connected to the camera head 13 via a signal line and a communication interface. The signal line between the camera head 13 and the CCU 51 is, for example, a high-speed transmission line capable of enabling bidirectional communication, such as an optical cable. The CCU 51 includes a processor such as a central processing unit (CPU) and a memory such as a random access memory (RAM) and comprehensively controls operation of the endoscope 10 and the display device 53. The CCU 51 may further include a frame memory for temporarily storing image signals and one or more graphics processing units (GPUs) that execute image processing. For example, the CCU 51 determines a disparity for each pixel (or some unit) on the basis of the disparity-determination image input from the camera head 13. For example, the determined disparity can be used for image processing such as generation of a stereoscopic image, extension of a depth of field, emphasis of a stereoscopic effect, or expansion of a dynamic range. The CCU 51 can output an image generated as a result of the image processing to the display device 53 for display or output the image to a recorder 65 for recording. A series of output images can form a moving image (video). The image processing executed in the CCU 51 may include, for example, general processing such as development and noise reduction. Further, the CCU 51 transmits a control signal to the camera head 13 to control drive of the camera head 13. The control signal can include, for example, information that specifies the imaging conditions described above.

The display device 53 displays the stereoscopic image on the basis of the input display image signals under the control of the CCU 51. The display device 53 may display the stereoscopic image by any method such as an active shutter method, a passive method, or a glassless method.

The light source device 55 includes, for example, an LED, a xenon lamp, a halogen lamp, a laser light source, or a light source corresponding to a combination thereof and supplies irradiation light with which the observation target is to be irradiated to the endoscope 10 via the light guide.

The arm control device 57 includes, for example, a processor such as a CPU and operates in accordance with a predetermined program to control drive of the arm portion 43 of the support arm device 40.

An input device 59 includes one or more input interfaces that accept user input to the medical image processing system 1. The user can input various pieces of information or input various instructions to the medical image processing system 1 via the input device 59. For example, the user may input setting information or other parameters described below via the input device 59. Further, for example, the user inputs an instruction to drive the arm portion 43, an instruction to change the imaging conditions (the type of irradiation light, a magnification, a focal distance, and the like) in the endoscope 10, an instruction to drive the energy treatment device 33, or the like via the input device 59.

The input device 59 may treat any type of user input. For example, the input device 59 may detect physical user input via a mechanism such as a mouse, a keyboard, a switch (e.g., a foot switch 69), or a lever. The input device 59 may detect touch input via a touchscreen. The input device 59 may be achieved in the form of a wearable device such as an eyeglass-type device or a head mounted display (HMD) and may detect a line of sight or gesture of the user. Further, the input device 59 may include a microphone capable of acquiring voice of the user and may detect an audio command via the microphone.

A treatment tool control device 61 controls drive of the energy treatment device 33 for treatment such as cauterization or incision of tissue or sealing of a blood vessel. A pneumoperitoneum device 63 secures a visual field observed by using the endoscope 10 and sends gas into the body cavity via the pneumoperitoneum tube 31 in order to inflate the body cavity of the patient 7 for the purpose of securing an operational space of the practitioner. A recorder 65 records various pieces of information regarding medical operation (e.g., one or more of setting information, image information, and measurement information from a vital sensor (not illustrated)) on a recording medium. A printer 67 prints various pieces of information regarding medical operation in some format such as text, an image, or a graph.

### [1-2. Description of Problem]

Determination of a disparity for generation of a stereoscopic image or another use in such a medical image processing system is typically performed on the basis of comparison between two images showing a common visual field from different points of sight. For example, according to a block matching method using two images from a stereo camera, a position of a certain block in one image having a small image having the highest similarity to a small image of a block in the other image is searched for, and a disparity is determined on the basis of a difference between the positions of the blocks obtained as a result of the search. In the present specification, the blocks used in such a search are referred to as “collation blocks”. The collation blocks generally have a rectangular shape.

However, in the field of medical image processing in many cases, the conventional block matching method has not provided sufficient disparity determination accuracy in a case of, for example, minute subjects such as a needle tool and a thread for use in suturing or ligation in surgical operation.

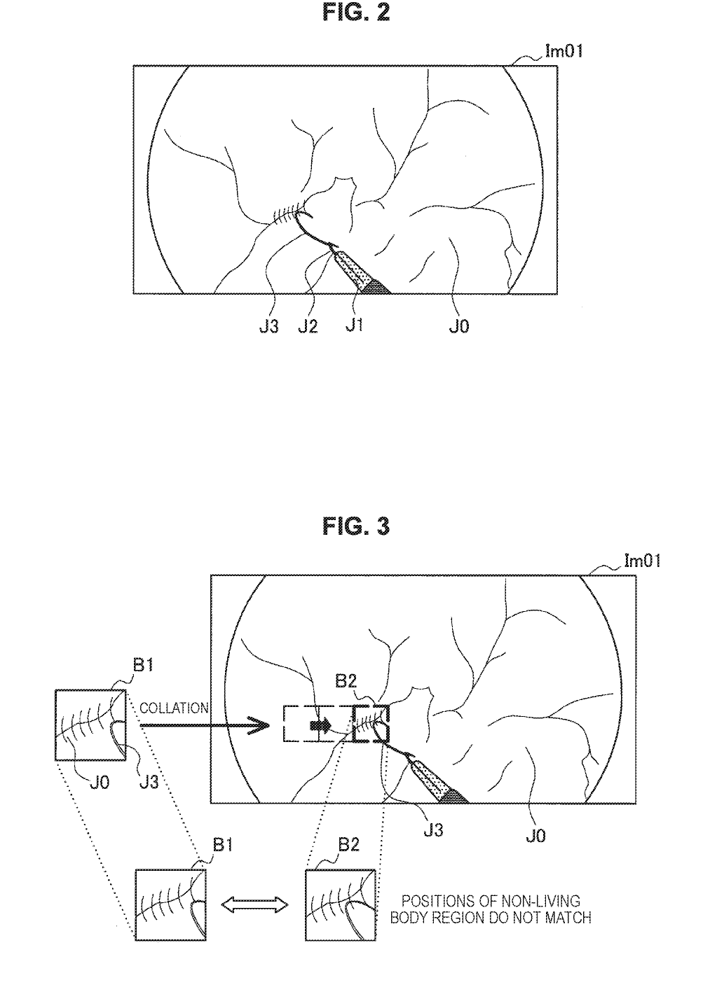

FIG. 2 is an explanatory view illustrating an example of a disparity-determination image showing a minute subject. When referring to FIG. 2, an image Im01 is exemplified, and the image Im01 can be a right-eye image between the right-eye image and a left-eye image forming the disparity-determination image. In the image Im01, a living body J0, a forceps J1, a needle tool J2, and a thread J3 appear as a subject. A region of the forceps J1 occupying the image Im01 is relatively smaller than that of the living body J0, and regions of the needle tool J2 and the thread J3 occupying the image Im01 are minute.

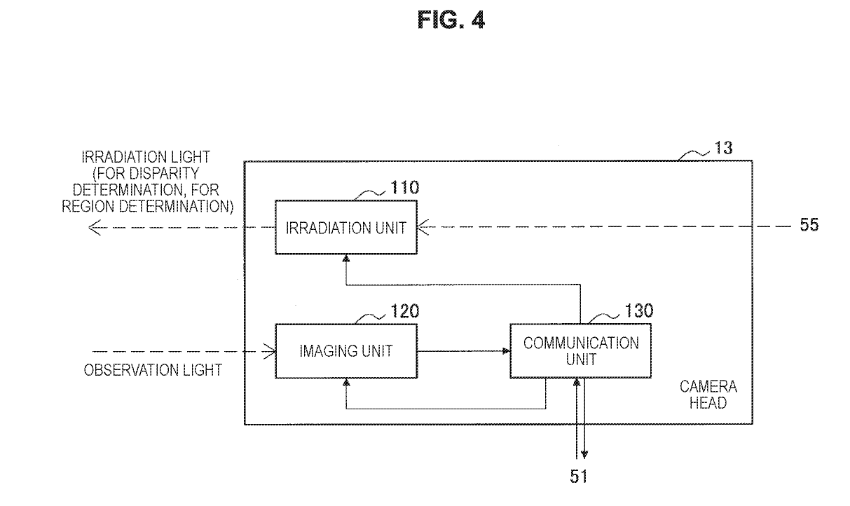

FIG. 3 is an explanatory diagram for describing accuracy of a disparity determined in accordance with an existing block matching method. A block B1 illustrated in a left part of FIG. 3 is, for example, a collation block cut from the left-eye image forming the disparity-determination image together with the image Im01 that is a right-eye image. The subjects J0 and J3 appear in the collation block B1. When a small image having the highest similarity to the collation block B1 (e.g., a sum total of differences between pixels is the smallest) is searched for in the image Im01 in accordance with the block matching method, the collation block B1 can match with, for example, a block B2 in the image Im01. Comparing the block B1 with the block B2, small images of the subject J0 corresponding to a background have sufficient similarity. Meanwhile, a position of the subject J3 in the collation block B1 does not match with a position of the subject J3 in the block B2. Such mismatch can occur for the following reason: disparities of the two subjects are different due to a difference between depths of those subjects, and a difference in the view of the subject J3 is not sufficiently considered in collation between the small images because the subject J3 is small and a larger subject J0 influences a result of the collation. However, in a medical scene such as surgical operation or diagnosis, in many cases, high visibility is generally demanded in a case of a minute subject or a subject smaller than a background, such as a medical instrument that is a non-living body (e.g., a needle tool and a thread for ligation). However, the existing method does not sufficiently satisfy such a demand. Embodiments described below of the technology of the present disclosure focus on such a disadvantage and improve an existing method to enhance accuracy of determination of a disparity for medical image processing.

### 2. First Embodiment

Among the constituent elements of the medical image processing system 1 exemplified in FIG. 1, in particular, the camera head 13 functioning as an imaging device and the CCU 51 functioning as an image processing device mainly relate to determination of a disparity and image processing based on the determined disparity. In view of this, in this section, specific configurations of those two devices according to an embodiment will be described in detail.

Note that, in the medical image processing system 1, those imaging device and image processing device are separately provided and are connected to each other via a signal line. However, the technology of the present disclosure is not limited to such an example. For example, a function of the image processing device described below may be mounted in a processor included in the imaging device. Further, the imaging device may record an image signal on a recording medium, and the image processing device may process the image signal read out from the recording medium (In this case, no signal line may exist between those two devices.).

### [2-1. Imaging Device]

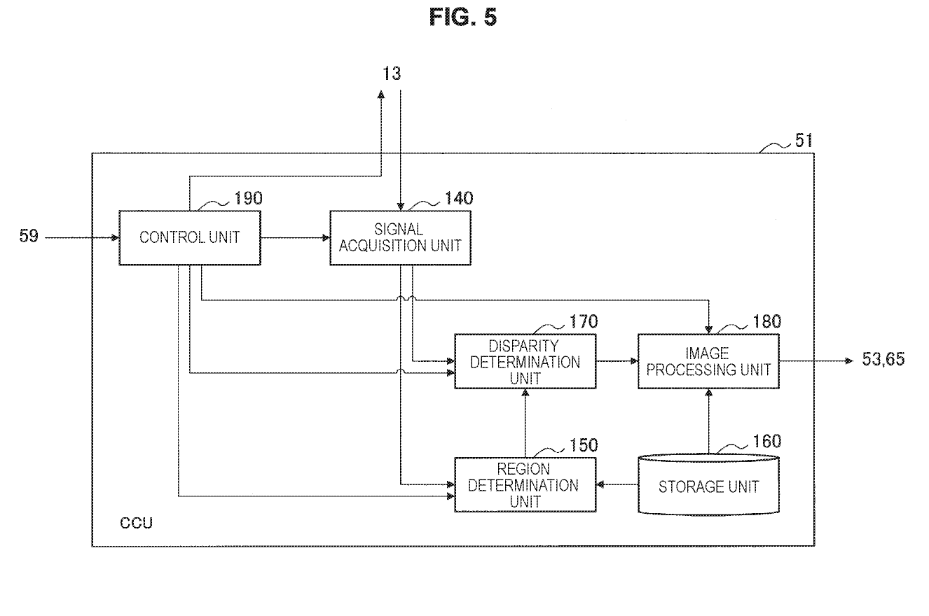

FIG. 4 is a block diagram illustrating an example of a configuration of the camera head 13 according to the first embodiment. When referring to FIG. 4, the camera head 13 includes an irradiation unit 110, an imaging unit 120, and a communication unit 130.

The irradiation unit 110 irradiates a subject in a visual field with irradiation light supplied from the light source device 55 via the light guide. The light emitted by the irradiation unit 110 may be, for example, visible light used for the purpose of capturing a disparity-determination image. Further, in a case where special light observation (e.g., fluorescence observation) is performed, disparity-determination irradiation light may be special light having a wavelength suitable for the type of observation.

Further, in the present embodiment, for the purpose of capturing a region-determination image, the irradiation unit 110 irradiates the same subject with light having a wavelength whose spectral characteristic of at least one of a living body and a non-living body is already known. Region-determination irradiation light may be the same or different from disparity-determination irradiation light. It is known that spectral characteristics (or spectral reflectance ratios) of living bodies can be generally written in several types of modes at most (Yuri Murakami, “Theory of Spectral Reflectance Estimation”, Journal of the Society of Photographic Science and Technology of Japan Vol. 65, 2002, No. 4, pp. 234-239). Spectral characteristics of non-living bodies can also be measured in advance by types and be written as spectral characteristic information. The region-determination irradiation light emitted by the irradiation unit 110 in the present embodiment has one or more wavelength components whose spectral characteristics are already known. In a case where region determination described below needs a plurality of (e.g., four or more) wavelength components, the irradiation unit 110 may irradiate a subject with rays of irradiation light having different wavelength patterns by a time division method. The wavelength (or wavelength pattern) and an irradiation timing of the irradiation light emitted from the irradiation unit 110 can be controlled in accordance with a control signal from the CCU 51.

The imaging unit 120 can typically include an optical system, an image sensor, and a drive system. The optical system of the imaging unit 120 collects observation light from the subject, the observation light being taken in through the tip of the lens barrel 11, toward the image sensor via a pair of lenses (also referred to as “lens unit”). The image sensor performs photoelectric conversion on the collected observation light and generates an image signal serving as an electric signal. For example, when disparity-determination irradiation light is emitted, an image signal of a disparity-determination image is generated as a result of capturing an image of reflected light thereof. The disparity-determination image includes a right-eye image and a left-eye image as described above. When region-determination irradiation light is emitted, an image signal of a region-determination image is generated as a result of capturing an image of reflected light thereof. The imaging unit 120 outputs those image signals generated by the image sensor to the communication unit 130. The drive system of the imaging unit 120 moves a movable member such as a zoom lens or focus lens of the optical system in accordance with a control signal from the CCU 51.

The communication unit 130 is a communication interface connected to the CCU 51 via a signal line. For example, the communication unit 130 transmits the above-mentioned image signals generated by the imaging unit 120 to the CCU 51.

Further, when a control signal is received from the CCU 51, the communication unit 130 outputs the received control signal to the irradiation unit 110 and the imaging unit 120.

### [2-2. Configuration Example of Image Processing Device]

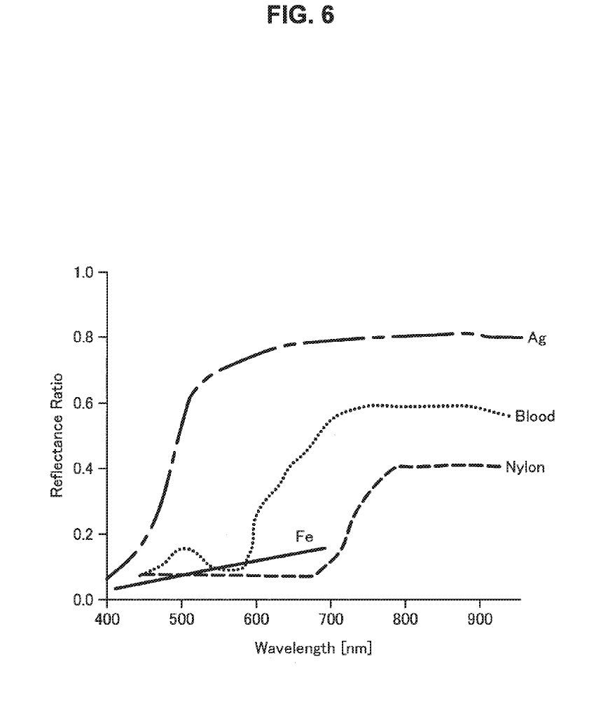

FIG. 5 is a block diagram illustrating an example of a configuration of the CCU 51 according to the first embodiment. When referring to FIG. 5, the CCU 51 includes a signal acquisition unit 140, a region determination unit 150, a storage unit 160, a disparity determination unit 170, an image processing unit 180, and a control unit 190.

### (1) Signal Acquisition Unit

The signal acquisition unit 140 acquires an image signal of a region-determination image and an image signal of a disparity-determination image, which are generated in the imaging unit 120 of the camera head 13. The region-determination image is a captured image showing the visual field that is the same as a visual field that the disparity-determination image shows. The signal acquisition unit 140 outputs the region-determination image to the region determination unit 150. Further, the signal acquisition unit 140 outputs the disparity-determination image to the disparity determination unit 170.

### (2) Region Determination Unit

The region determination unit 150 determines a non-living body region in an observed visual field by using the region-determination image input from the signal acquisition unit 140. In the present embodiment, the region-determination image is an image having one or more wavelength components serving as references of analysis of spectral characteristics and generated by capturing an image of observation light from a subject as described above. Spectral characteristic information stored on the storage unit 160 writes a spectral characteristic of a target that is at least one of a living body and a non-living body. The region determination unit 150 analyzes a spectral characteristic of the subject on the basis of the region-determination image by using the spectral characteristic information, thereby determining a non-living body region in the observed visual field.

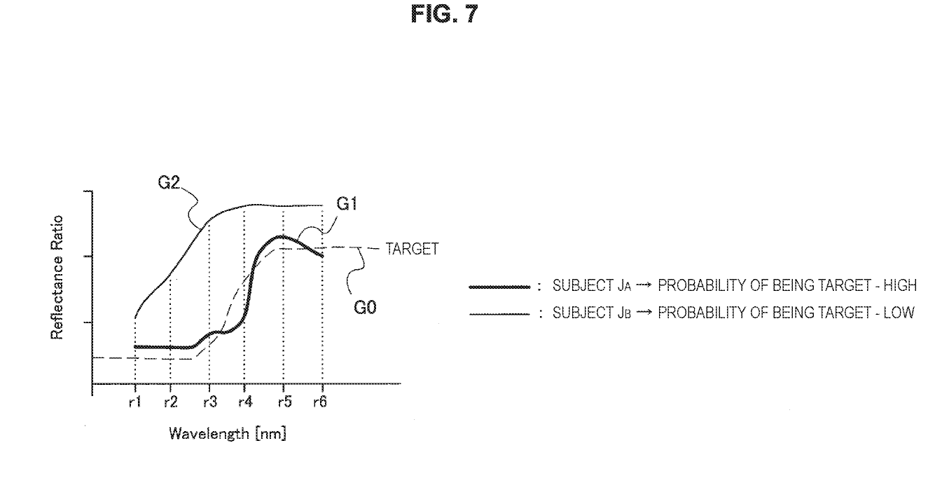

FIG. 6 is an explanatory diagram for describing spectral characteristics of several materials. FIG. 6 is a graph showing spectral characteristics of four different types of materials. A horizontal axis of the graph indicates a wavelength and a vertical axis thereof indicates a reflectance ratio. A graph of a solid line exemplarily indicates a spectral characteristic of iron, a graph of a broken line exemplarily indicates a spectral characteristic of nylon that is a kind of synthetic resin, a graph of an alternate long and short dash line exemplarily indicates a spectral characteristic of silver, and a graph of a dotted line exemplarily indicates a spectral characteristic of blood. In particular, in a case where an image of the inside of a living body is captured, the spectral characteristic of blood greatly influences a spectral characteristic of the inside of the body and can therefore be treated as a representative spectral characteristic of a living body. Nylon is one of materials used for medical threads. Stainless containing iron as a main component is one of materials used for medical needle tools. Silver is also one of materials used for medical needle tools. As understood from those graphs, a subject is irradiated with light having one or more specified wavelength components at an already-known strength, and a strength of each wavelength component of reflected light thereof is measured, and then a result of the measurement is compared with the already-known spectral characteristics of the materials. This makes it possible to determine whether the subject is a living body or a non-living body. Further, in a case where the subject is a non-living body, it is also possible to determine which type of medical instrument the subject is by using a similar method. The strength of each wavelength component of the reflected light is indicated by a pixel value of each pixel of the region-determination image for each signal component. Note that not only the spectral characteristics of the four types of materials exemplified in FIG. 6 but also, for example, spectral characteristics of various materials used for medical instruments, such as glass and rubber, may be utilized.

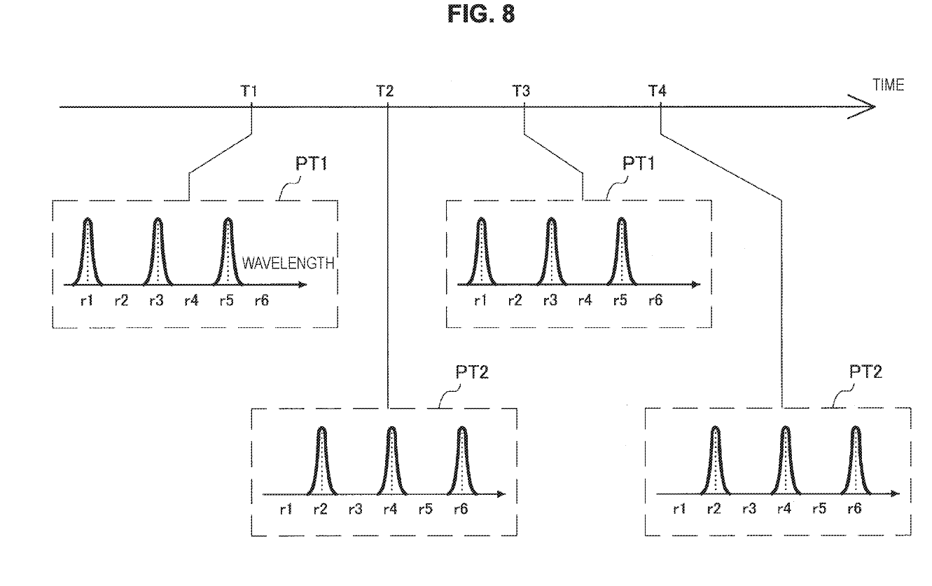

FIG. 7 is an explanatory diagram for describing an example of analysis of spectral characteristics of subjects. A graph GO shown in FIG. 7 shows a spectral characteristic of a certain already-known non-living body (target). The spectral characteristic herein is treated as a set of reflectance ratios of respective wavelength components at six sample wavelengths r1, r2, r3, r4, r5, and r6. A graph G1 shows a spectral characteristic of a subject J_{A} existing at a first pixel position, which is recognized on the basis of a pixel value of one or more region-determination images. The region determination unit 150 compares the spectral characteristic shown by the graph G1 with the already-known spectral characteristic of the target shown by the graph G0 and can therefore calculate a probability (or likelihood) that the subject J_{A} is a target. For example, in a case where a sum total of errors between the spectral characteristics is zero, the probability that the subject J_{A} is a target is the highest. As the error between the spectral characteristics is increased, the probability that the subject J_{A} is a target is reduced. A graph G2 shows a spectral characteristic of a subject J_{B} existing at a second pixel position, which is recognized on the basis of a pixel value of one or more region-determination images. The region determination unit 150 compares the spectral characteristic shown by the graph G2 with the already-known spectral characteristic of the target shown by the graph G0 and can therefore calculate a probability that the subject J_{B} is a target.

In the example of FIG. 7, the graph G1 has higher similarity to the graph G0 than the graph G2 (A sum total of differences between reflectance ratios at each sample wavelength is smaller.). Therefore, in this case, the region determination unit 150 can determine that the first pixel position belongs to a non-living body region corresponding to the target, whereas the second pixel position does not belong to the non-living body region corresponding to the target. The region determination unit 150 calculates, for example, the probability (a target likelihood or similarity of spectral characteristics) for each pixel and compares the calculated probability with a threshold and can therefore perform region determination for each pixel. In a case where the target is a non-living body, a pixel showing a probability exceeding the threshold can belong to the non-living body region and a pixel showing a probability below the threshold can belong to a living body region. In a case where the target is a living body (e.g., blood), a pixel showing a probability exceeding the threshold belongs to the living body region and a pixel showing a probability below the threshold belongs to the non-living body region.

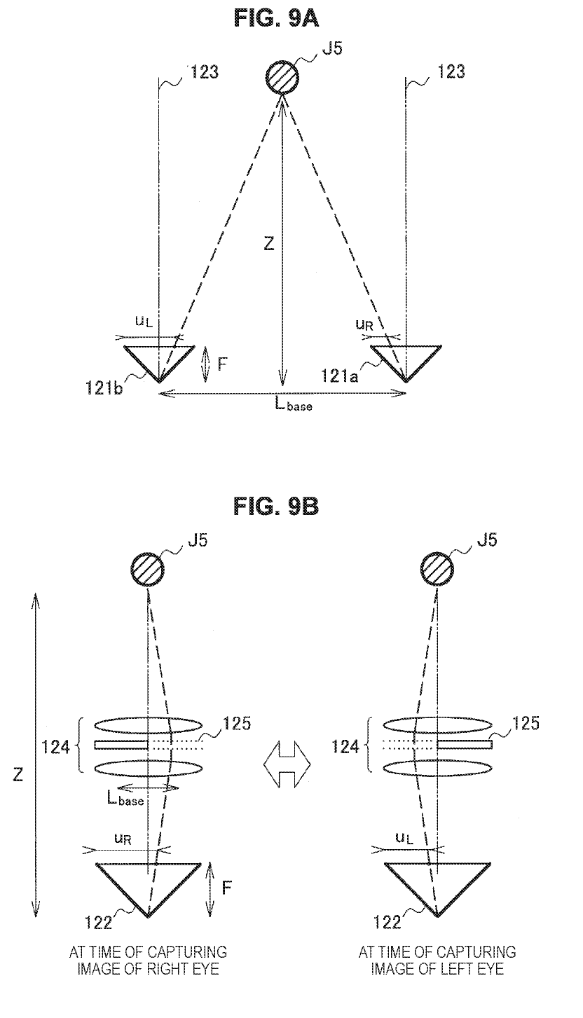

Accuracy of region determination based on analysis of a spectral characteristic of a subject is increased as sample wavelengths serving as references of analysis are increased. However, a normal imaging device captures an image of observation light simultaneously containing three color components at most. In view of this, in a certain example, it is advantageous to capture an image of a wavelength pattern of irradiation light while switching a plurality of patterns by the time division method. FIG. 8 is an explanatory diagram for describing an example of such switching of wavelength patterns. When referring to FIG. 8, light having a wavelength pattern PT1 is emitted at a time T1, and an image of reflected light is captured. The wavelength pattern PT1 includes sample wavelengths r1, r3, and r5.

Then, light having a wavelength pattern PT2 is emitted at a time T2, and an image of reflected light is captured. The wavelength pattern PT2 includes sample wavelengths r2, r4, and r6. Then, light having the wavelength pattern PT1 is emitted again at a time T3, and an image of reflected light is captured. Then, light having the wavelength pattern PT2 is emitted again at a time T4, and an image of reflected light is captured. The region determination unit 150 can recognize a spectral characteristic of a subject based on the six sample wavelengths r1 to r6 by using, for example, values of six signal components in total of the two region-determination images captured at the times T1 and T2, respectively. Similarly, the region determination unit 150 can recognize a spectral characteristic of the next frame based on the six sample wavelengths r1 to r6 by using values of six signal components in total of the region-determination images captured at the times T3 and T4, respectively.

The region determination unit 150 may segment the region-determination image into a living body region and one or more non-living body regions. For example, in a case where spectral characteristics of a plurality of types of non-living bodies (e.g., two or more of forceps, needle tools, and threads) are already known, the region determination unit 150 may calculate a probability that each pixel belongs to each type of non-living body region and segment the region-determination image into a living body region and one or more non-living body regions on the basis of the calculated probability. Instead of this, in a case where a spectral characteristic of a single non-living body (or living body) is already known, the region determination unit 150 may execute, for example, a segmentation technique such as a graph cut method on the basis of a calculated probability that each pixel belongs to a non-living body region (or a living body region), thereby segmenting the region-determination image into a living body region and one or more non-living body regions. In a case where it is determined that a plurality of non-living body regions exist in the region determination image, a collation block in each non-living body region can be individually set in determination of a disparity described below.

### (3) Storage Unit

The storage unit 160 stores information for various types of processing executed in the CCU 51. For example, the storage unit 160 stores spectral characteristic information to be used by the region determination unit 150 for region determination. The spectral characteristic information includes a data set of reflectance ratios at respective sample wavelengths, which shows an already-known spectral characteristic of each of one or more targets. Each target can be at least one of a living body and a non-living body. A target whose spectral characteristic is to be analyzed may be switched between a plurality of possible targets on the basis of the type of operation to be performed in the medical image processing system 1 (e.g., a surgical form of surgical operation) or the type of instrument to be used. The storage unit 160 may store target setting information that associates the type of operation or the instrument to be used with the target whose spectral characteristic is to be analyzed (e.g., a thread and a needle tool can be associated with ligation operation).

### (4) Disparity Determination Unit

The disparity determination unit 170 determines a disparity by using the disparity-determination image input from the signal acquisition unit 140. More specifically, in the present embodiment, the disparity determination unit 170 determines at least a disparity to be applied to the non-living body region by using not only the disparity-determination image but also a result of the determination of the non-living body region performed by the region determination unit 150. A disparity can be typically determined by searching for, at each pixel position (or each pixel block) of one of the right-eye image and the left-eye image forming the disparity-determination image, a corresponding point in the other image in accordance with the block matching method and calculating a difference in horizontal position from the found corresponding point.

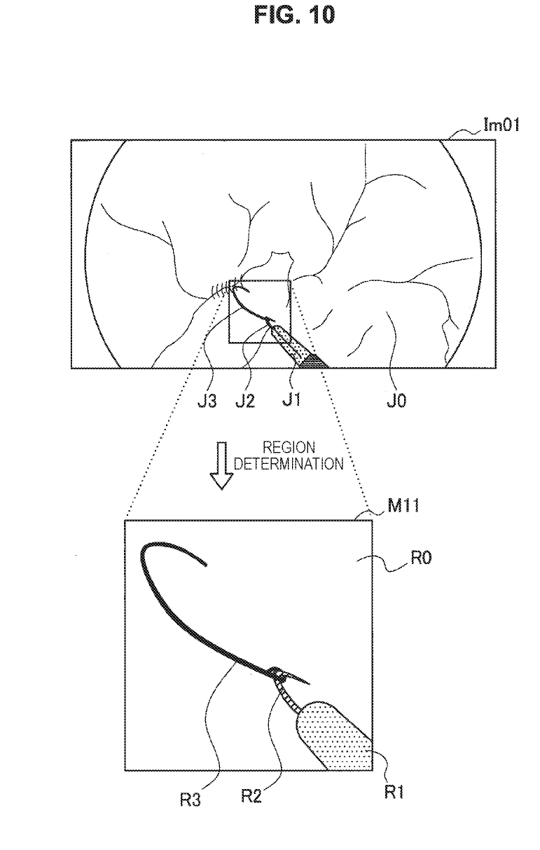

FIG. 9A is an explanatory diagram for describing disparity determination using a compound-eye stereo camera. When referring to FIG. 9A, a right-eye image sensor 121a and a left-eye image sensor 121b are each illustrated as an inverted triangle. An upper side of each inverted triangle corresponds to an imaging surface of the image sensor and a bottom vertex corresponds to a focal point. In FIG. 2A, F denotes a focal distance, and L_{base} denotes a base length between the image sensors (e.g., a distance between two optical centers 123). In the example of FIG. 9A, a subject J5 exists at a position of a distance Z from the base line between the right-eye image sensor 121a and the left-eye image sensor 121b. In the present specification, this distance Z will be referred to as “depth”. The subject J5 appears at a horizontal position u_{R} on the imaging surface of the right-eye image sensor 121a. Further, the subject J5 appears at a horizontal position u_{L} on the imaging surface of the left-eye image sensor 121b. In a case where the use of a pinhole camera model is presupposed for simplicity of explanation, a disparity d based on the right-eye image sensor 121a is given by the following expression:

[Math. 1]

d=u_{L}−u_{R} (1)

Further, when the depth Z has a variable value, the disparity d(Z) serving as a function of the depth Z can be expressed by the following expression with the use of the base length L_{base} and the focal distance F:

$$[ Math . 2 ]$$

$$d ( Z ) = {L}_{base} \cdot \frac{F}{Z} \qquad ( 2 )$$

FIG. 9B is an explanatory diagram for describing disparity determination using a monocular stereo camera. When referring to FIG. 9B, a single image sensor 122 is illustrated as an inverted triangle. Further, not only a pair of lenses 124 but also a shutter 125 capable of selectively shielding a right half or a left half of the lenses is disposed in front of the image sensor 122. When an image of the right eye is captured, the shutter 125 shields the left half of the lenses and an image of observation light collected through the right half of the lenses is captured by the image sensor 122. When an image of the left eye is captured, the shutter 125 shields the right half of the lenses and an image of observation light collected through the left half of the lenses is captured by the image sensor 122. By capturing the images while temporally repeating such switching of the shutter, sequences of the right-eye image and the left-eye image are generated by the image sensor 122. The base length L_{base} between the right-eye image and the left-eye image can be defined as, for example, a distance between a center of balance of the right half of a pupil (which corresponds to an image of a diaphragm of the optical system) at a position of the shutter 125 and a center of balance of the left half thereof. Also in the monocular stereo camera, the disparity d(Z) can be expressed by Expression (2) described above as the function of the depth Z of the subject by using the base length L_{base} and the focal distance F.

As described above, it is possible to derive the disparity d(Z) by using the block matching method on the basis of the right-eye image and the left-eye image, regardless of the system of the stereo camera, and the derived disparity d(Z) can be further used to calculate the depth Z of the subject in a case where, for example, the base length and the focal distance are already known. Note that, in a case where two subjects are different, actual disparities of those subjects are also different from each other. In addition, in a case where a collation block used at the time of block matching includes such two subjects having different actual depths, a disparity obtained as a result of determination is closer to an actual disparity of a subject that makes a larger contribution to an index of determination. In view of this, in the present embodiment, in order to improve accuracy of determination of a disparity of a medical instrument that is a minute non-living body or a non-living body smaller than a background in many cases, the disparity determination unit 170 adaptively sets a collation block on the basis of a result of determination of the non-living body region and collates a first disparity-determination image (one of the right-eye image and the left-eye image) with a second disparity-determination image (the other of the right-eye image and the left-eye image) by using the set collation block.

For example, the disparity determination unit 170 sets a size of the collation block depending on a size or shape of the non-living body region determined by the region determination unit 150. Further, the disparity determination unit 170 sets a weight of each pixel in each collation block, which is to be used for collation, depending on the shape of the non-living body region. The disparity determination unit 170 may set a weight of a pixel corresponding to the non-living body region in the collation block to a first value (e.g., 1) and a weight of a pixel corresponding to the living body region in the collation block to a second value (e.g., zero). The second value can mean that a pixel value at a corresponding pixel position is not added to a collation determination index or is added thereto as an extremely small value. The collation determination index may be, for example, a weighted sum of an absolute value of a difference between the pixels. Instead of this, the disparity determination unit 170 may set the weight of each pixel in the collation block depending on a probability that the pixel belongs to the non-living body region. The probability that each pixel belongs to the non-living body region can be already calculated when region determination is performed by the region determination unit 150.

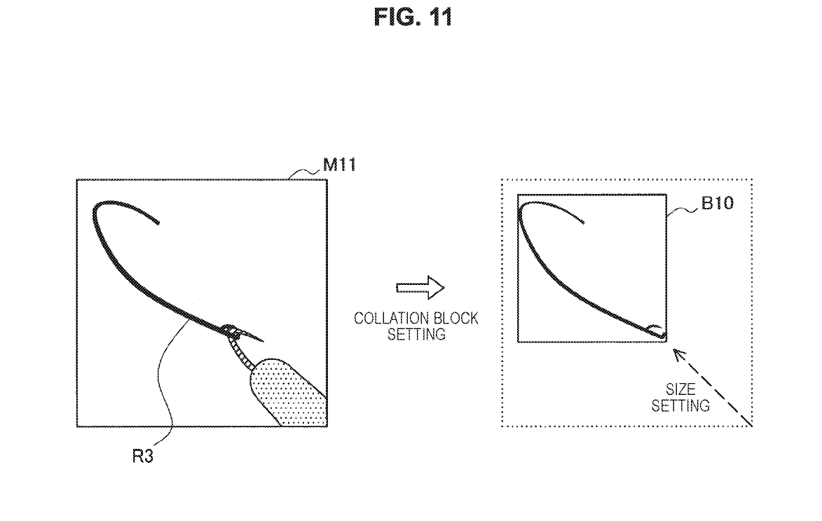

Hereinafter, a method for determining a disparity to be applied to a non-living body region will be described in detail with reference to FIGS. 10 to 12B.

FIG. 10 is an explanatory diagram for describing an example of a result of determination of a non-living body region using a region-determination image. An upper part of FIG. 10 illustrates the disparity-determination image Im01 described with reference to FIG. 2 again. A result of region determination based on a region-determination image showing the visual field that is the same as a visual field that the disparity-determination image Im01 shows may be shown by, for example, binary mask information indicating whether or not each pixel belongs to a non-living body region at each pixel position by using binary values (true (1): belonging to the non-living body region, false (zero): not belonging to the non-living body region, or the like). Instead of this, the result of the region determination may be shown by multivalue mask information indicating which type of non-living body region or living body region each pixel belongs to at each pixel position by using multivalues (e.g., zero: living body, 1: thread, 2: needle tool, 3: forceps, or the like). In the example of FIG. 10, content of multivalue mask information M11 showing the result of the region determination is partially enlarged for clarity. For example, the multivalue mask information M11 shows a position and shape of a forceps region R1 serving as a non-living body region with a dot-halftone pattern. Further, the multivalue mask information M11 shows a position and shape of a needle tool region R2 serving as a non-living body region with an oblique-line hatching pattern. Further, the multivalue mask information M11 shows a position and shape of a thread region R3 serving as a non-living body region with a black shading pattern.

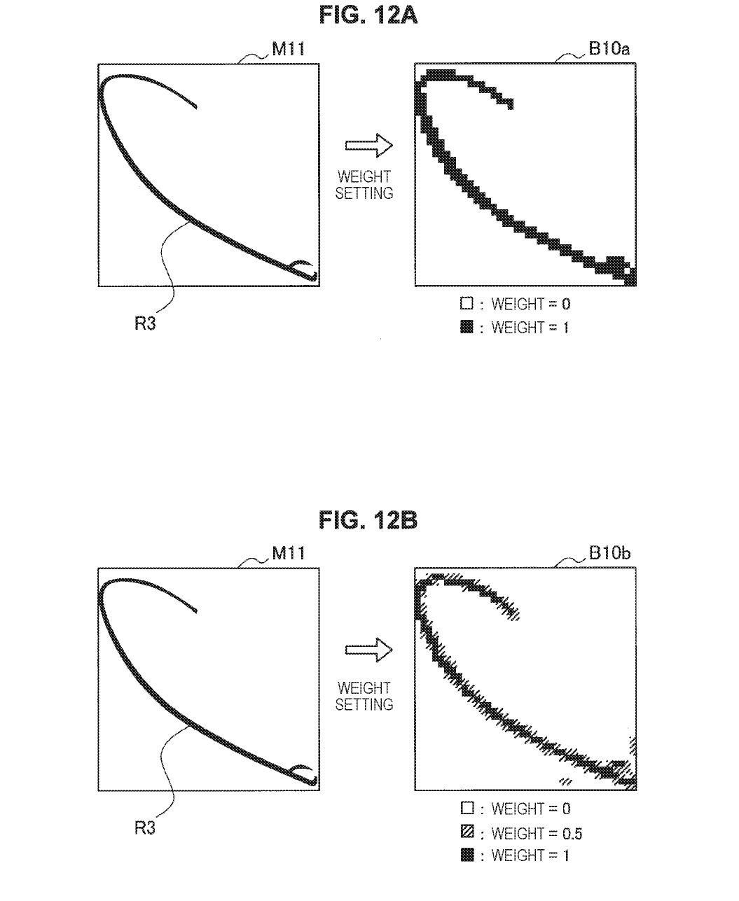

FIG. 11 is an explanatory diagram for describing an example of a collation block that is set on the basis of a result of determination of a non-living body region. A left part of FIG. 11 illustrates the multivalue mask information M11 again. For example, the disparity determination unit 170 sets at least a size of a collation block B10 to be used for determination of a disparity of the thread region R3 depending on a size and shape of the thread region R3 shown by the multivalue mask information M11. The collation block may typically have a square or oblong shape, i.e., a rectangular shape. A length of each side of the rectangular shape is set so that the collation block can cover the whole or most of the non-living body region or cover a characteristic part of the non-living body region. In the example of FIG. 11, the disparity determination unit 170 sets the collation block B10 to be used for determination of the disparity of the thread region R3 as the smallest square that can cover the whole thread region R3. Note that the collation block may have a shape other than the rectangular shape. Further, the shape of the collation block may be set depending on the shape of the non-living body region.

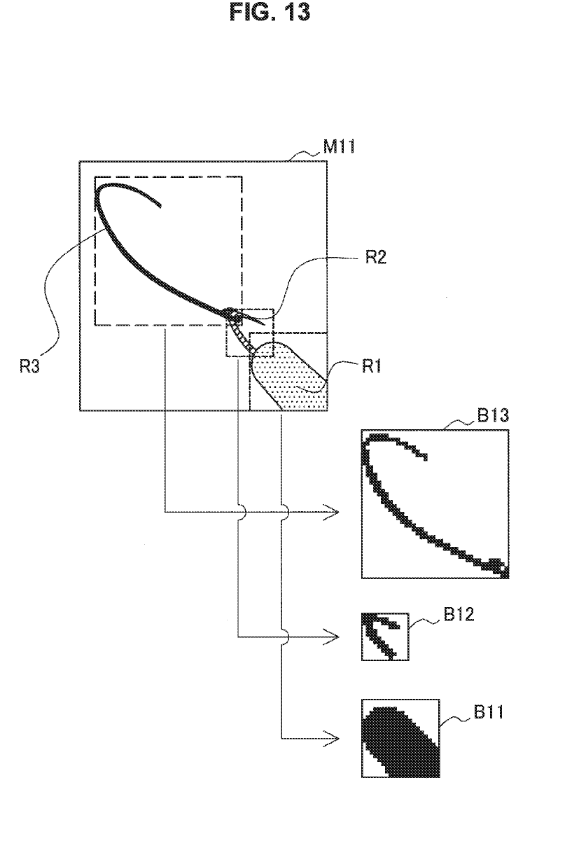

FIG. 12A is an explanatory diagram for describing a first example of setting a weight to a collation block. In the first example illustrated in FIG. 12A, the disparity determination unit 170 sets a weight of a pixel corresponding to the thread region R3 in a collation block B10a to 1 and sets a weight of a pixel that does not correspond to the thread region R3 in the collation block B10a (mainly, a pixel corresponding to a living body region) to zero. By setting a weight of a pixel in the collation block which does not correspond to a target region of determination of a disparity (a region of a subject where highly accurate determination of a disparity is desired) to zero as described above, it is possible to substantially ignore such a pixel in block matching. That is, even in a case where the target region is minute, it is possible to perform accurate block matching on the target region, without changing the collation determination index due to an influence of a subject (having a different disparity) around the target region.

FIG. 12B is an explanatory diagram for describing a second example of setting a weight to a collation block. In the second example illustrated in FIG. 12B, the disparity determination unit 170 sets a weight of a pixel having a high probability of belonging to the thread region R3 in a collation block B10b to 1, sets a weight of a pixel having an intermediate probability of belonging to the thread region R3 to 0.5, and sets a weight of a pixel having a low probability of belonging to the thread region R3 to zero. By setting a weight at each pixel position to be added to the collation determination index of block matching depending on a probability that each pixel belongs to a target region as described above, it is possible to derive an optimal result of block matching also in consideration of certainty of region determination.

In a case where the region determination unit 150 determines that a plurality of non-living body regions exist in a visual field, the disparity determination unit 170 may individually set a collation block in each non-living body region and determine a disparity to be applied to each non-living body region by using the set collation block.

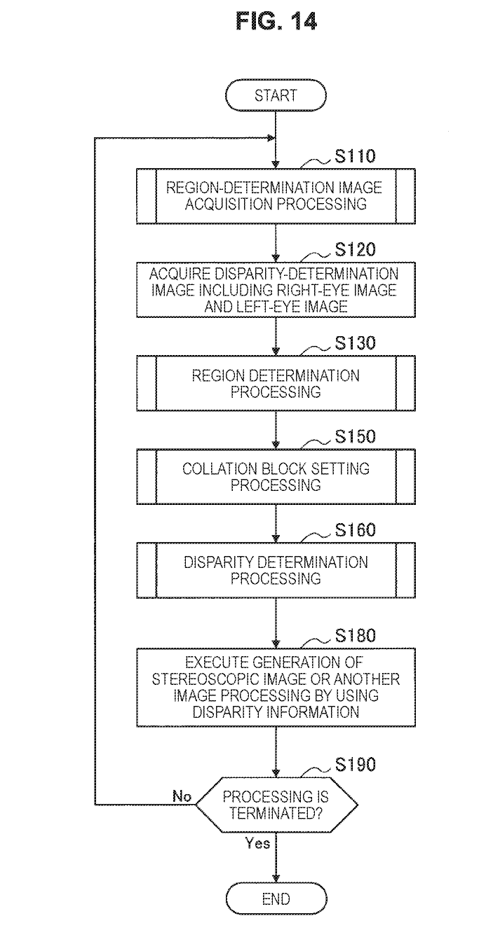

FIG. 13 is an explanatory diagram for describing an example of setting a plurality of collation blocks. FIG. 13 illustrates the multivalue mask information M11 described with reference to FIG. 10 again. The multivalue mask information M11 shows the positions and shapes of the forceps region R1, the needle tool region R2, and the thread region R3 serving as non-living body regions. For example, those non-living body regions R1, R2, and R3 can be distinguished from one another as a result of segmentation of the non-living body region in the region determination unit 150. The disparity determination unit 170 can set a collation block B11, a collation block B12, and a collation block B13 on the basis of the forceps region R1, the needle tool region R2, and the thread region R3, respectively, shown by such multivalue mask information M11. Then, the disparity determination unit 170 can determine a disparity of the forceps by block matching using the collation block B11 with high accuracy. Similarly, the disparity determination unit 170 can determine a disparity of the needle tool by block matching using the collation block B12 with high accuracy and can determine a disparity of the thread by block matching using the collation block B13 with high accuracy.

The disparity determination unit 170 determines a disparity at each pixel position (or pixel block position) by using a collation block that is set on the basis of a result of determination of a non-living body region as described above and outputs disparity information indicating the determined disparity to the image processing unit 180. Further, the disparity determination unit 170 outputs the disparity-determination image to the image processing unit 180.

### (5) Image Processing Unit

The image processing unit 180 executes image processing based on the disparity indicated by the disparity information input from the disparity determination unit 170. For example, the image processing unit 180 may generate a stereoscopic image corresponding to the observed visual field on the basis of the disparity indicated by the disparity information. More specifically, for example, the image processing unit 180 shifts a horizontal position of a pixel in accordance with the disparity indicated by the disparity information in one of the right-eye image and the left-eye image input from the disparity determination unit 170, thereby generating a stereoscopic image. The image processing unit 180 may interpolate pixel information of a defective region whose pixel information is defective due to the shift of the horizontal position of the pixel on the basis of adjacent pixels having no defect. In the present embodiment, accuracy of determination of a disparity of a non-living body is improved on the basis of a result of determination of a non-living body region. This makes it possible to provide a more accurate stereoscopic effect of a minute non-living body subject or a non-living body subject smaller than a background in the generated stereoscopic image.

Further, the image processing unit 180 may execute extended depth of field (EDOF) processing on the basis of the disparity indicated by the disparity information. More specifically, the image processing unit 180 calculates, for example, a depth of each pixel on the basis of the disparity indicated by the disparity information. Then, the image processing unit 180 executes deblurring of a captured image (at least one of disparity-determination images) with a filter configuration depending on the calculated depth and can therefore simulatively extend a depth of field of the captured image. In the present embodiment, accuracy of determination of a disparity of a non-living body is improved on the basis of a result of determination of a non-living body region. This makes it possible to execute such extended depth of field processing more accurately.

Further, the image processing unit 180 may execute stereoscopic effect emphasis processing on the basis of the disparity indicated by the disparity information. More specifically, for example, the image processing unit 180 corrects the disparity indicated by the disparity information by multiplying the disparity by a predetermined coefficient or adding (or subtracting) a predetermined offset to (or from) the disparity and generates a stereoscopic image by using the corrected disparity. In the present embodiment, accuracy of determination of a disparity of a non-living body is improved on the basis of a result of determination of a non-living body region. This makes it possible to emphasize a stereoscopic effect of a minute non-living body subject or a non-living body subject smaller than a background more accurately and reduce a risk that an unnatural stereoscopic image is displayed.

The image processing unit 180 may further execute, for example, another image processing such as high dynamic range processing, noise reduction, white balance adjustment, and an increase in resolution at an arbitrary timing before or after the above-mentioned image processing. Further, the disparity determined by the disparity determination unit 170 may be utilized in those types of image processing. Then, the image processing unit 180 outputs an image signal (of, e.g., the stereoscopic image or the image whose depth of field has been extended) which has been subjected to such image processing to the display device 53 or the recorder 65.

### (6) Control Unit

The control unit 190 controls operation of the endoscope 10 on the basis of user input and setting information detected by the input device 59 so that an image is captured and is displayed as the user desires. For example, when capturing of an image is started, the control unit 190 causes the irradiation unit 110 of the camera head 13 to emit region-determination irradiation light in one or more wavelength patterns determined in advance or dynamically selected at a timing determined in advance or dynamically selected and causes the signal acquisition unit 140 to acquire an image signal of a region-determination image through capturing the image in the imaging unit 120. Further, the control unit 190 causes the irradiation unit 110 to emit disparity-determination irradiation light and causes the signal acquisition unit 140 to acquire an image signal of a disparity-determination image through capturing the image in the imaging unit 120. Further, the control unit 190 causes the region determination unit 150 to determine a non-living body region on the basis of an already-known spectral characteristic of one or more targets and causes the disparity determination unit 170 to determine a disparity to be applied to the non-living body region by using a result of the determination. Then, the control unit 190 causes the image processing unit 180 to execute image processing such as generation of a stereoscopic image or extension of a depth of field on the basis of the use of the system. The control unit 190 may cause the user to specify the type of operation (e.g., surgical form such as heart bypass operation or gastrectomy or the type of procedure such as ligation or suturing) via a user interface. In this case, the control unit 190 can select a wavelength pattern of region-determination irradiation light and a target whose spectral characteristic is to be analyzed on the basis of the type of operation specified by the user. Further, the control unit 190 may cause the user to specify a medical instrument to be used (e.g., a needle tool and a thread) via the user interface. In this case, the control unit 190 can select a wavelength pattern of region-determination irradiation light and a target whose spectral characteristic is to be analyzed on the basis of the instrument specified by the user. Further, the control unit 190 may dynamically switch the type of image processing to be executed in the image processing unit 180 depending on user input or another setting. The user input herein may be any type of input such as physical input, touch input, gesture input, or audio input described regarding the input device 59.

### [2-3. Flow of Processing]

In this section, examples of a flow of processing that can be executed by the camera head 13 and the CCU 51 in the above embodiment will be described with reference to several flowcharts. Note that, although a plurality of processing steps are shown in the flowcharts, those processing steps do not necessarily need to be executed in order shown in the flowcharts. Several processing steps may be executed in parallel. Further, an additional processing step may be employed, or part of the processing steps may be omitted. The same applies to description of embodiments in the following sections.

### (1) Whole Flow

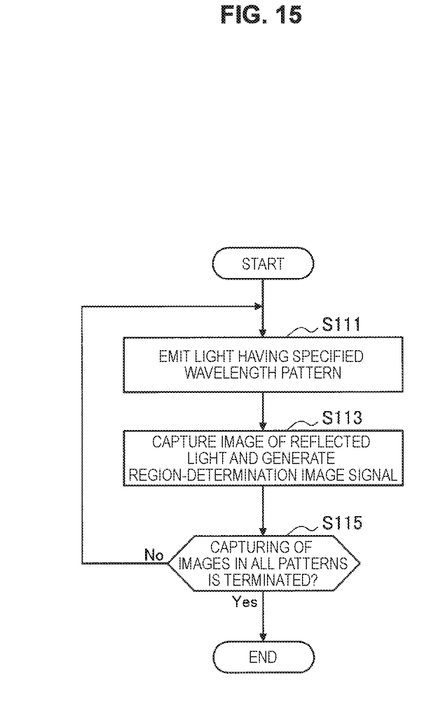

FIG. 14 is a flowchart showing an example of the whole flow of image processing according to the first embodiment. When referring to FIG. 14, first, the signal acquisition unit 140 acquires an image signal of a region-determination image generated in the imaging unit 120 of the camera head 13 (Step S110). An example of a more detailed flow of this region-determination image acquisition processing executed herein will be further described below. Further, the signal acquisition unit 140 acquires an image signal of a disparity-determination image including a right-eye image and a left-eye image (Step S120).

Then, the region determination unit 150 executes region determination processing for determining a non-living body region in an observed visual field by using the region-determination image supplied from the signal acquisition unit 140

(Step S130). An example of a more detailed flow of the region determination processing executed herein will be further described below.

Then, the disparity determination unit 170 executes collation block setting processing on the basis of a result of the region determination processing in Step S130 and sets a collation block to be used for determination of a disparity of the non-living body region in the observed visual field (Step S150). Several examples of a more detailed flow of the collation block setting processing executed herein will be further described below.

Then, the disparity determination unit 170 executes disparity determination processing by using the collation block that has been set in the collation block setting processing in Step S150 and determines at least a disparity to be applied to the non-living body region of the disparity-determination image (Step S160). An example of a more detailed flow of the disparity determination processing executed herein will be further described below.

Then, the image processing unit 180 executes generation of a stereoscopic image or another image processing by using disparity information generated as a result of the disparity determination processing in Step S160 (Step S180).

Steps S110 to S180 described above are repeated until a termination condition of the image processing is satisfied (Step S190). For example, when user input to give an instruction to terminate the processing is detected via the input device 59, the above processing is terminated.

### (2) Region-Determination Image Acquisition Processing

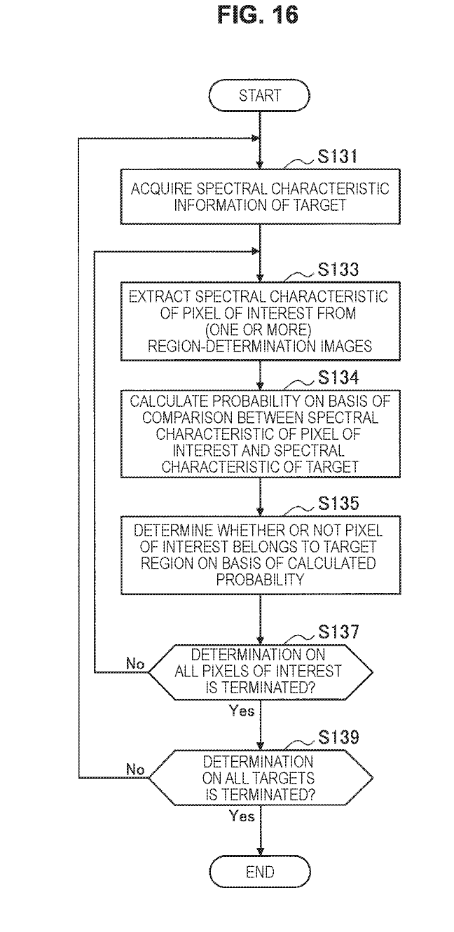

FIG. 15 is a flowchart showing an example of the detailed flow of the region-determination image acquisition processing shown in FIG. 14. When referring to FIG. 15, first, the control unit 190 transmits a control signal to the camera head 13 to cause the camera head 13 to emit light having a specified wavelength pattern from the irradiation unit 110 (Step S111). Further, the control unit 190 causes the imaging unit 120 to capture an image of reflected light of the emitted light and causes the imaging unit 120 to generate an image signal of the region-determination image (Step S113). The control unit 190 repeats emission of irradiation light and and capturing of an image of reflected light in Steps S111 and S113 until capturing of images in all necessary wavelength patterns is terminated (Step S115). Then, when capturing of images (for one frame) in all wavelength patterns is terminated, the region-determination image acquisition processing shown in FIG. 15 is terminated.

### (3) Region Determination Processing

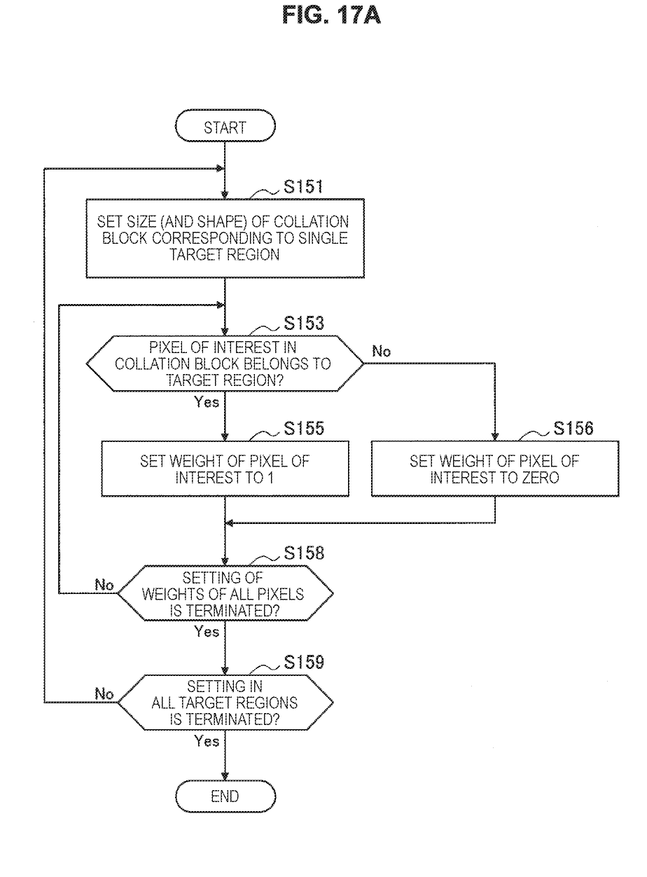

FIG. 16 is a flowchart showing an example of the detailed flow of the region determination processing shown in FIG. 14. When referring to FIG. 16, first, the region determination unit 150 acquires spectral characteristic information indicating an already-known spectral characteristic of a target that can be selected by the control unit 190 from the storage unit 160 (Step S131).

Then, the region determination unit 150 extracts a spectral characteristic of one of pixels (hereinafter, referred to as “pixel of interest”) successively selected in the image from the region-determination image (Step S133). In a case where the spectral characteristic is extracted as reflectance ratios of three or less wavelength samples, only a single region-determination image may be referred to herein. In a case where the spectral characteristic is extracted as reflectance ratios of four or more wavelength samples, a plurality of region-determination images acquired by capturing images a plurality of times in different wavelength patterns can be referred to. Then, the region determination unit 150 calculates a probability that the pixel of interest belongs to a target region on the basis of comparison between the spectral characteristic of the extracted pixel of interest and the already-known spectral characteristic of the target (Step S134). Then, the region determination unit 150 determines whether or not the pixel of interest belongs to the target region on the basis of the calculated probability (Step S135).

The region determination unit 150 repeats Steps S133 to S135 described above until determination on all pixels of interest is terminated (Step S137). Further, the region determination unit 150 repeats the above-mentioned determination until a region determination result of all of one or more targets is derived (Step S139). Then, when (binary or multivalue) mask information indicating the region determination result of all the targets is generated, the region determination processing shown in FIG. 16 is terminated.

### (4) Collation Block Setting Processing—First Example

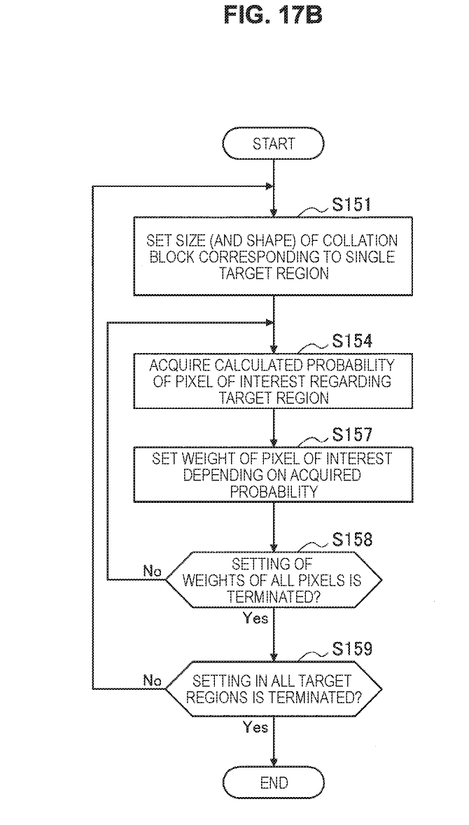

FIG. 17A is a flowchart showing a first example of the detailed flow of the collation block setting processing shown in FIG. 14. When referring to FIG. 17A, first, the disparity determination unit 170 sets a size of a collation block corresponding to a target region that is one of one or more non-living body regions indicated by the region determination result depending on a size or shape of the target region (Step S151). The shape of the collation block may be set depending on the shape of the corresponding target region or may be fixed.

Then, the disparity determination unit 170 determines whether or not a pixel of interest in the collation block belongs to the target region (Step S153) and sets a weight of a pixel of interest belonging to the target region to 1 (Step S155) and sets a weight of a pixel of interest that does not belong to the target region to zero (Step S156). The disparity determination unit 170 repeats such setting of a weight until setting of weights of all the pixels in the collation block is terminated (Step S158). When this repetition is terminated, setting of a collation block corresponding to a single target region is terminated.

Further, in a case where a plurality of target regions exist, the disparity determination unit 170 repeats Steps S151 to S158 described above until setting of collation blocks corresponding to all the target regions is terminated (Step S159). When this repetition is terminated, setting of collation blocks corresponding to all the target regions indicated by the region determination result is terminated.

### (5) Collation Block Setting Processing—Second Example

FIG. 17B is a flowchart showing a second example of the detailed flow of the collation block setting processing shown in FIG. 14. When referring to FIG. 17B, first, the disparity determination unit 170 sets a size (or size and shape) of a collation block corresponding to a single target region depending on a size or shape of the target region (Step S151).

Then, the disparity determination unit 170 acquires a probability of a pixel of interest in the collation block regarding the target region, which is calculated by the region determination unit 150 (a probability that the pixel of interest belongs to the target region) (Step S154). Then, the disparity determination unit 170 sets a weight of the pixel of interest depending on the acquired probability (Step S157). The disparity determination unit 170 repeats such setting of a weight until setting of weights of all the pixels in the collation block is terminated (Step S158). When this repetition is terminated, setting of a collation block corresponding to a single target region is terminated.

Further, in a case where a plurality of target regions exist, the disparity determination unit 170 repeats Steps S151 to S158 described above until setting of collation blocks corresponding to all the target regions is terminated (Step S159). When this repetition is terminated, setting of collation blocks corresponding to all the target regions indicated by the region determination result is terminated.

### (6) Disparity Determination Processing

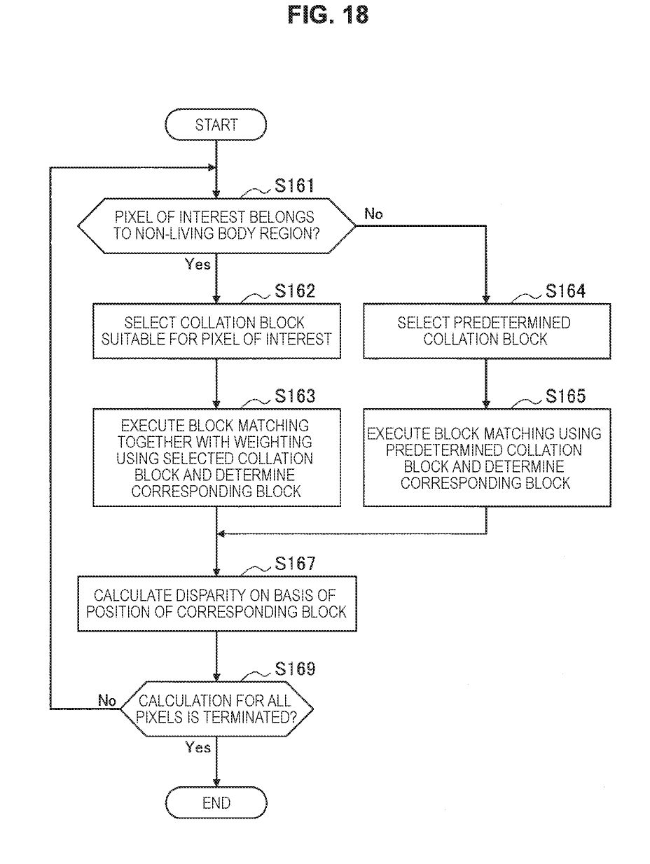

FIG. 18 is a flowchart showing an example of the detailed flow of the disparity determination processing shown in FIG. 14. When referring to FIG. 18, first, the disparity determination unit 170 determines whether or not a pixel of interest in the disparity-determination image belongs to a non-living body region (Step S161).

In a case where the pixel of interest belongs to the non-living body region, the disparity determination unit 170 selects a collation block suitable for the pixel of interest from the one or more collation blocks that have been set in the collation block setting processing (Step S162). The collation block selected herein may be a collation block corresponding to the non-living body region in which a probability that the pixel of interest belongs is the highest. Then, the disparity determination unit 170 executes block matching together with weighting using the selected collation block and determines a corresponding block corresponding to the pixel of interest (Step S163).

In contrast, in a case where the pixel of interest does not belong to the non-living body region, the disparity determination unit 170 selects a predetermined collation block (Step S164). The predetermined collation block can be, for example, a block having a general shape (e.g., rectangular shape) and a weight that is uniform in all pixels. Then, the disparity determination unit 170 executes block matching by using the predetermined collation block and determines a corresponding block corresponding to the pixel of interest (Step S165).

Then, the disparity determination unit 170 calculates a disparity of the pixel of interest (e.g., as a difference in a horizontal direction between a reference position of the corresponding block and a pixel position of the pixel of interest) on the basis of a position of the corresponding block determined as a result of the block matching (Step S167). The disparity determination unit 170 repeats Steps S161 to S167 described above until calculation of disparities of all pixels in the disparity-determination image is terminated (Step S169). The disparity determination unit 170 outputs disparity information indicating the disparity at each pixel position determined as described above to the image processing unit 180.

### 3. Second Embodiment

In the first embodiment, a non-living body region in a visual field is determined on the basis of analysis of a spectral characteristic of a subject using a region-determination image. In a second embodiment that will be described in this section, a non-living body region is determined more easily on the basis of a signal strength of a region-determination image captured as a far infrared image, instead of analysis of a spectral characteristic.

### [3-1. Configuration Example of Imaging Device]

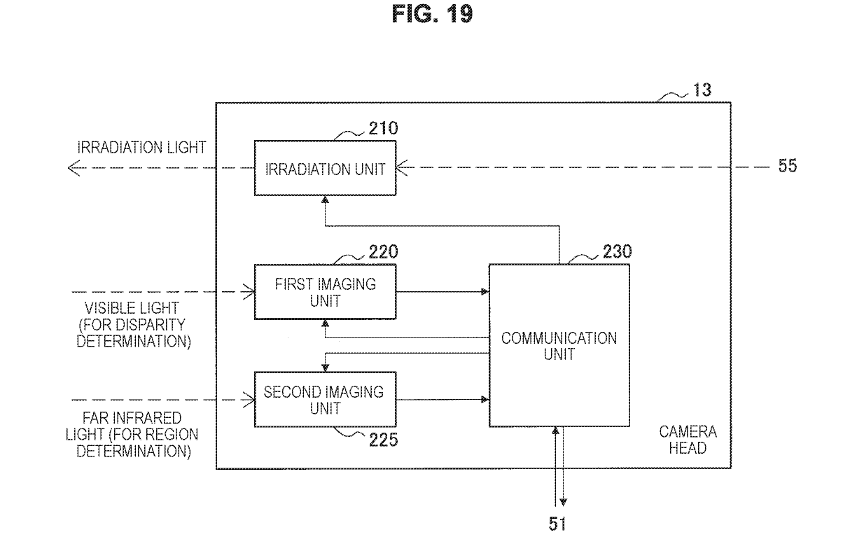

FIG. 19 is a block diagram illustrating an example of a configuration of the camera head 13 according to the second embodiment. When referring to FIG. 19, the camera head 13 includes an irradiation unit 210, a first imaging unit 220, a second imaging unit 225, and a communication unit 230.

The irradiation unit 210 irradiates a subject in a visual field with irradiation light supplied from the light source device 55 via the light guide. The light emitted by the irradiation unit 210 may be, for example, visible light used for the purpose of capturing a disparity-determination image. Further, in a case where special light observation (e.g., fluorescence observation) is performed, disparity-determination irradiation light may be special light having a wavelength suitable for the type of observation. In the present embodiment, the irradiation unit 210 does not emit region-determination irradiation light.

The first imaging unit 220, as well as the imaging unit 120 according to the first embodiment, performs photoelectric conversion on observation light from a subject, the observation light being taken in through the tip of the lens barrel 11, in the image sensor and generates an image signal of a disparity-determination image. The disparity-determination image includes a right-eye image and a left-eye image. Then, the first imaging unit 220 outputs the generated image signal to the communication unit 230.

In the present embodiment, the second imaging unit 225 is an imaging device that captures an image of far infrared light instead of visible light. Far infrared light is, for example, infrared light belonging to a wavelength range having a wavelength longer than a wavelength boundary of 1μm. The second imaging unit 225 can typically include an optical system including a filter that allows far infrared light radiating from a living body to pass therethrough and an image sensor having sensitivity to a wavelength range including a far infrared region. The second imaging unit 225 captures an image of far infrared light from the subject and generates an image signal of the far infrared image. Then, the second imaging unit 225 outputs the generated image signal to the communication unit 230.

The communication unit 230 is a communication interface connected to the CCU 51 via a signal line. For example, the communication unit 230 transmits the image signal of the disparity-determination image generated by the first imaging unit 220 to the CCU 51. Further, the communication unit 230 transmits the image signal of the region-determination image generated by the second imaging unit 225 to the CCU 51. Further, when a control signal is received from the CCU 51, the communication unit 230 outputs the received control signal to the irradiation unit 210, the first imaging unit 220, and the second imaging unit 225.

### [3-2. Configuration Example of Image Processing Device]

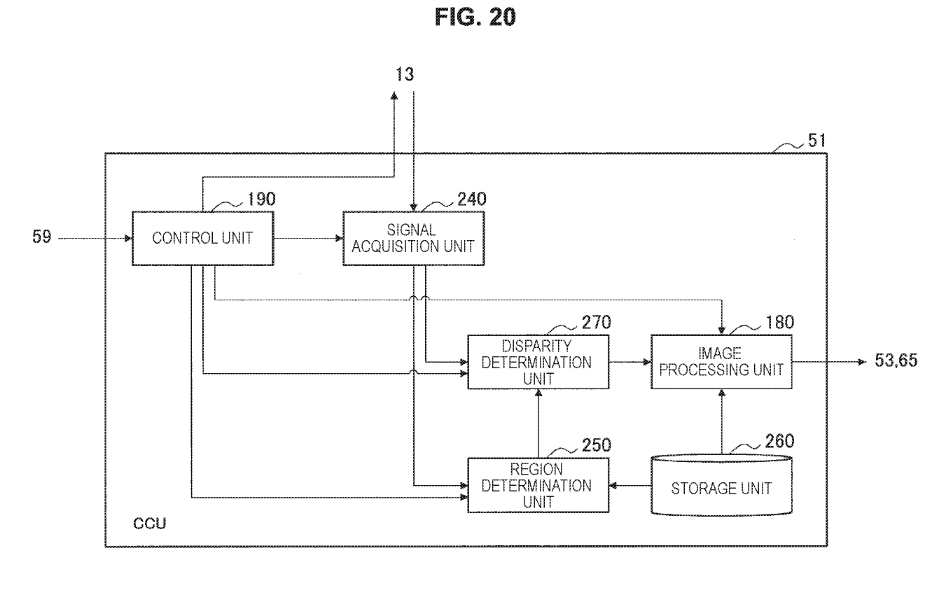

FIG. 20 is a block diagram illustrating an example of a configuration of the CCU 51 according to the second embodiment. When referring to FIG. 20, the CCU 51 includes a signal acquisition unit 240, a region determination unit 250, a storage unit 260, a disparity determination unit 270, an image processing unit 180, and a control unit 190.

### (1) Signal Acquisition Unit

The signal acquisition unit 240 acquires the image signal of the region-determination image (far infrared image) generated by the second imaging unit 225 of the camera head 13. Further, the signal acquisition unit 240 acquires the image signal of the disparity-determination image (visible light image) generated by the first imaging unit 220 of the camera head 13. Then, the signal acquisition unit 240 outputs the region-determination image to the region determination unit 250 and outputs the disparity-determination image to the disparity determination unit 270.

### (2) Region Determination Unit

The region determination unit 250 determines a non-living body region in an observed visual field by using the region-determination image input from the signal acquisition unit 240. In the present embodiment, the region-determination image is a far infrared image generated by capturing an image of far infrared light radiating from the subject as described above. Generally, living bodies such as organs and an abdominal wall radiate far infrared light, whereas medical instruments such as a thread, a needle tool, and a forceps hardly radiate far infrared light. In view of this, the region determination unit 250 compares a pixel value of each pixel in the region-determination image with a threshold defined in advance and can therefore determine which one of a living body region and a non-living body region each pixel belongs to. More specifically, the region determination unit 250 can determine that a pixel indicating a pixel value below the threshold in the region-determination image belongs to the non-living body region and determine that a pixel indicating a pixel value exceeding the threshold belongs to the living body region.

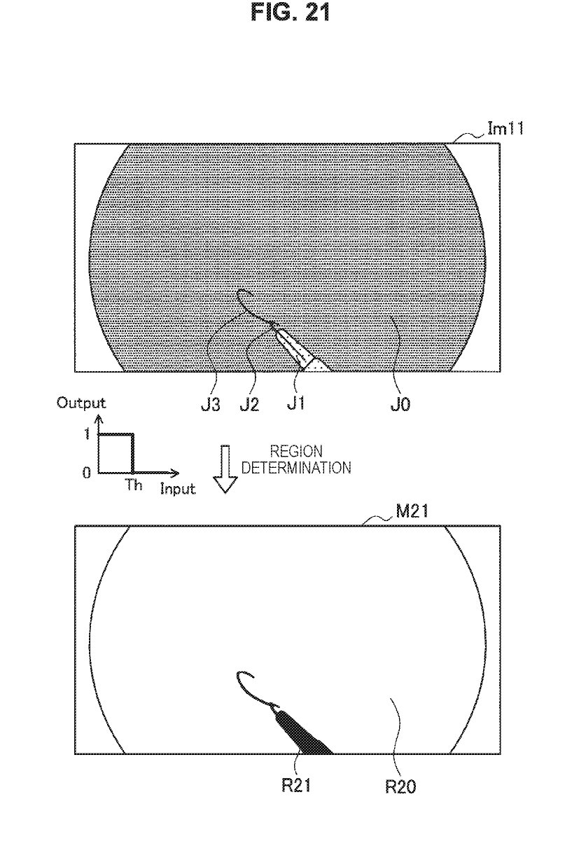

FIG. 21 is an explanatory diagram for describing another example of a result of determination of a non-living body region using a region-determination image. An upper part of FIG. 21 illustrates a region-determination image Im11 that is a far infrared image. A subject J0 appearing in the region-determination image Im11 is a living body radiating far infrared light, and therefore a pixel value of a region corresponding to the subject J0 indicates a relatively high value. In contrast, subjects J1, J2, and J3 appearing in the region-determination image Im11 are non-living bodies that hardly radiate far infrared light, and therefore pixel values of regions corresponding to the subjects J1, J2, and J3 indicate relatively low values. Results of such region determination based on comparison between the pixel values in the region-determination image Im11 and the threshold are shown by mask information M21 illustrated in a lower part of FIG. 21. The mask information M21 divides the whole image region into a living body region R20 and a non-living body region R21 (e.g., with a binary value of each pixel).

The region determination unit 250 may execute, for example, a segmentation technique such as a graph cut method, thereby segmenting the illustrated single non-living body region R21 into a plurality of non-living body regions. Further, in a case where noise appears because an image signal level of the far infrared image is weak, the region determination unit 250 may execute threshold determination for generating mask information after filtering for noise reduction.

### (3) Storage Unit

The storage unit 260 stores information for various types of processing executed in the CCU 51. For example, the storage unit 260 stores threshold information to be used by the region determination unit 250 for region determination.

### (4) Disparity Determination Unit

The disparity determination unit 270 determines a disparity by using the disparity-determination image input from the signal acquisition unit 240. More specifically, the disparity determination unit 270, as well as the disparity determination unit 170 according to the first embodiment, determines at least a disparity to be applied to the non-living body region by using not only the disparity-determination image but also a result of the determination of the non-living body region. In the present embodiment, the non-living body region is determined by the region determination unit 250 by using the region-determination image that is a far infrared image as described above. The disparity determination unit 270 may set a size of a collation block depending on a size or shape of the non-living body region determined by the region determination unit 250. Further, the disparity determination unit 270 may set a weight of each pixel in the collation block, which is to be used for collation, depending on the shape of the non-living body region. Typically, a weight of a pixel corresponding to the non-living body region in the collation block can be set to a first value (e.g., 1), and a weight of a pixel corresponding to the living body region in the collation block can be set to a second value (e.g., zero). The second value can mean that a pixel value at a corresponding pixel position is not added to a collation determination index or is added thereto as an extremely small value. In a case where the region determination unit 250 determines that a plurality of non-living body regions exist in the visual field, the disparity determination unit 270 may individually set a collation block in each non-living body region and determine a disparity to be applied to each non-living body region by using the set collation block. Further, the disparity determination unit 270 determines a disparity at each pixel position (or pixel block position) by using the collation block adaptively set in the non-living body region and by using a predetermined collation block in the living body region. Then, the disparity determination unit 270 outputs disparity information indicating a result of the determination to the image processing unit 180. Further, the disparity determination unit 270 outputs the disparity-determination image to the image processing unit 180. The disparity information generated by the disparity determination unit 270 may be used in the image processing unit 180 for various purposes such as generation of a stereoscopic image and extension of a depth of field as in the first embodiment.

### [3-3. Flow of Processing]

In this section, examples of a flow of processing that can be executed by the camera head 13 and the CCU 51 in the above-mentioned embodiment will be described with reference to several flowcharts.

### (1) Whole Flow

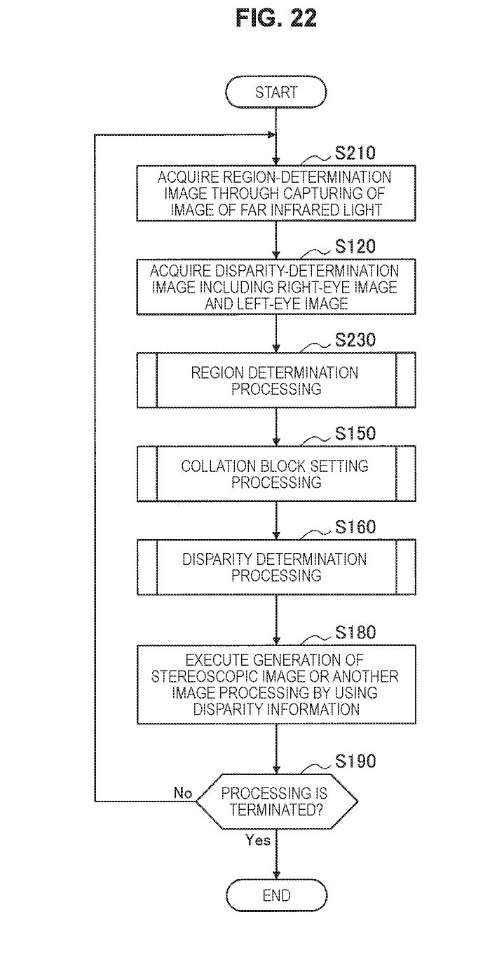

FIG. 22 is a flowchart showing an example of the whole flow of image processing according to the second embodiment. When referring to FIG. 22, first, the signal acquisition unit 240 acquires an image signal of a region-determination image through capturing the image in the second imaging unit 225 of the camera head 13 as an image of far infrared light (Step S210). Further, the signal acquisition unit 240 acquires an image signal of a disparity-determination image including a right-eye image and a left-eye image (Step S120).

Then, the region determination unit 250 executes region determination processing for determining a non-living body region in an observed visual field by using the region-determination image supplied from the signal acquisition unit 240 (Step S230). An example of a more detailed flow of the region determination processing executed herein will be further described below.

Then, the disparity determination unit 270 executes collation block setting processing on the basis of a result of the region determination processing in Step S230 and sets a collation block to be used for determination of a disparity of the non-living body region in the observed visual field (Step S150). The collation block setting processing executed herein may be similar to the processing described in the first embodiment.

Then, the disparity determination unit 270 executes disparity determination processing by using the collation block that has been set in the collation block setting processing in Step S150 and determines at least a disparity to be applied to the non-living body region of the disparity-determination image (Step S160). The disparity determination processing executed herein may be similar to the processing described in the first embodiment.

Then, the image processing unit 180 executes generation of a stereoscopic image or another image processing by using disparity information generated as a result of the disparity determination processing in Step S160 (Step S180).

Steps S210 to S180 described above are repeated until a termination condition of the image processing is satisfied (Step S190). For example, when user input to give an instruction to terminate the processing is detected via the input device 59, the above processing is terminated.

### (2) Region Determination Processing

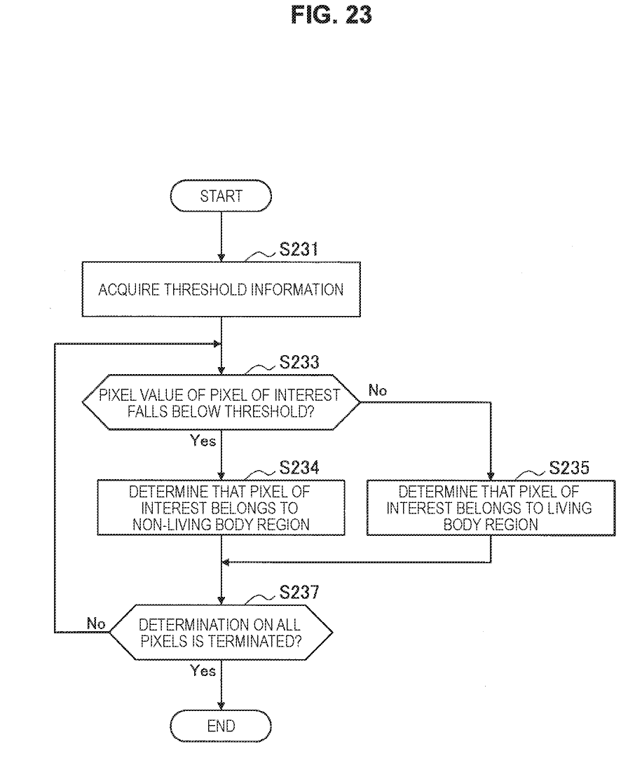

FIG. 23 is a flowchart showing an example of the detailed flow of the region determination processing shown in FIG. 22. When referring to FIG. 23, first, the region determination unit 250 acquires threshold information that can be defined in advance from the storage unit 260 (Step S231).

Then, the region determination unit 250 determines whether or not a pixel value of a pixel of interest falls below a threshold indicated by the threshold information acquired in Step S231 (Step S233). In a case where the pixel value of the pixel of interest falls below the threshold, the region determination unit 250 determines that the pixel of interest belongs to the non-living body region (Step S234). In contrast, in a case where the pixel value of the pixel of interest does not fall below the threshold, the region determination unit 250 determines that the pixel of interest belongs to the living body region (Step S235).

The region determination unit 250 repeats Steps S233 to S235 described above until determination on all pixels is terminated (Step S237). Then, when mask information indicating the region determination result of all the pixels is generated, the region determination processing shown in FIG. 23 is terminated.

### 4. Third Embodiment

In a third embodiment that will be described in this section, a region-determination image is also a far infrared image. However, in the third embodiment, an image of reflected light of far infrared light emitted toward a subject is captured.

### [4-1. Configuration Example of Imaging Ddevice]

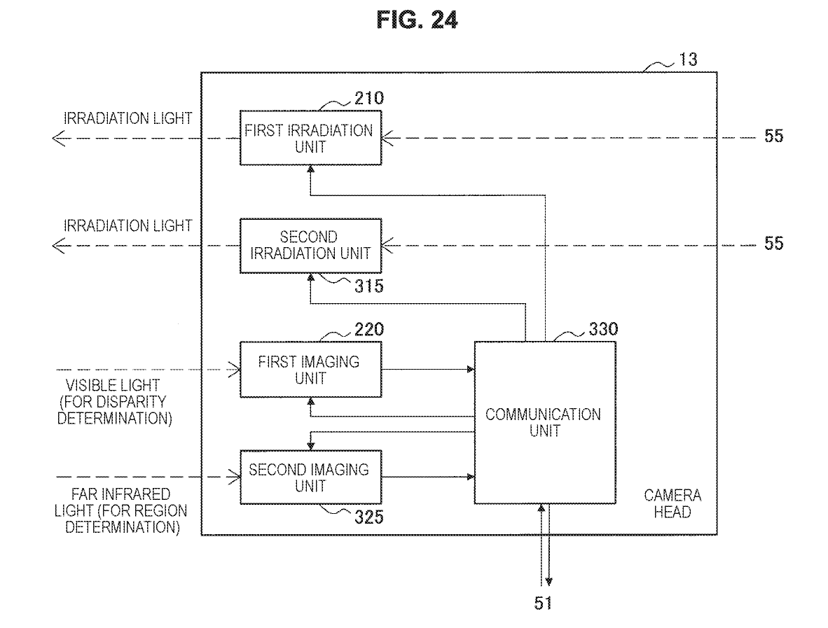

FIG. 24 is a block diagram illustrating an example of a configuration of the camera head 13 according to the third embodiment. When referring to FIG. 24, the camera head 13 includes a first irradiation unit 210, a second irradiation unit 315, a first imaging unit 220, a second imaging unit 325, and a communication unit 330.

The second irradiation unit 315 irradiates a subject in an observed visual field with far infrared light. The far infrared light emitted by the second irradiation unit 315 may be infrared light having any wavelength belonging to a far infrared region and used for the purpose of capturing a region-determination image.

The second imaging unit 325 is an imaging device that captures an image of far infrared light. The second imaging unit 325 can typically include an optical system including a filter that allows reflected light of far infrared light emitted by the second irradiation unit 315 to pass therethrough and an image sensor having sensitivity to a wavelength range including a far infrared region. The second imaging unit 325 captures an image of far infrared light reflected by the subject and generates an image signal of the far infrared image. Then, the second imaging unit 325 outputs the generated image signal to the communication unit 330.

The communication unit 330 is a communication interface connected to the CCU 51 via a signal line. For example, the communication unit 330 transmits the image signal of the disparity-determination image generated by the first imaging unit 220 to the CCU 51. Further, the communication unit 330 transmits the image signal of the region-determination image generated by the second imaging unit 325 to the CCU 51. Further, when a control signal is received from the CCU 51, the communication unit 330 outputs the received control signal to the first irradiation unit 210, the second irradiation unit 315, the first imaging unit 220, and the second imaging unit 325.

### [4-2. Configuration Example of Image Processing Device]

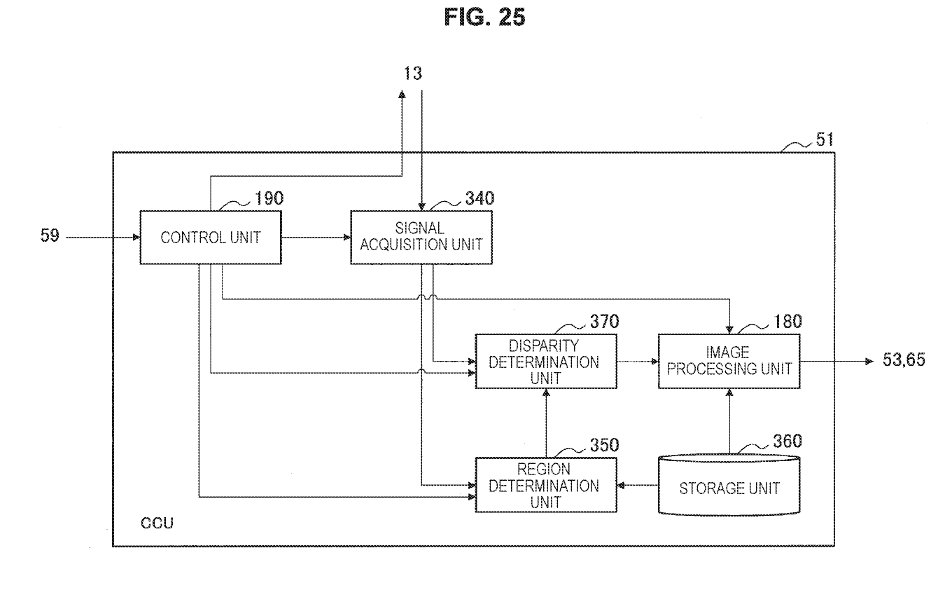

FIG. 25 is a block diagram illustrating an example of a configuration of the

CCU 51 according to the third embodiment. When referring to FIG. 25, the CCU 51 includes a signal acquisition unit 340, a region determination unit 350, a storage unit 360, a disparity determination unit 370, an image processing unit 180, and a control unit 190.

### (1) Signal Acquisition Unit

The signal acquisition unit 340 acquires the image signal of the region-determination image (far infrared image) generated by the second imaging unit 325 of the camera head 13. Further, the signal acquisition unit 340 acquires the image signal of the disparity-determination image (visible light image) generated by the first imaging unit 220 of the camera head 13. Then, the signal acquisition unit 340 outputs the region-determination image to the region determination unit 350 and outputs the disparity-determination image to the disparity determination unit 370.

### (2) Region Determination Unit

The region determination unit 350 determines a non-living body region in an observed visual field by using the region-determination image input from the signal acquisition unit 340. In the present embodiment, the region-determination image is a far infrared image generated by capturing an image of reflected light of far infrared light emitted toward the subject as described above. Generally, living bodies such as organs and an abdominal wall absorb far infrared light, whereas medical instruments such as a forceps including metal reflect far infrared light. In view of this, the region determination unit 350 compares a pixel value of each pixel in the region-determination image with a threshold defined in advance and can therefore determine which one of a living body region and a non-living body region each pixel belongs to. More specifically, the region determination unit 350 can determine that a pixel indicating a pixel value exceeding the threshold in the region-determination image belongs to the non-living body region and determine that a pixel indicating a pixel value below the threshold belongs to the living body region.

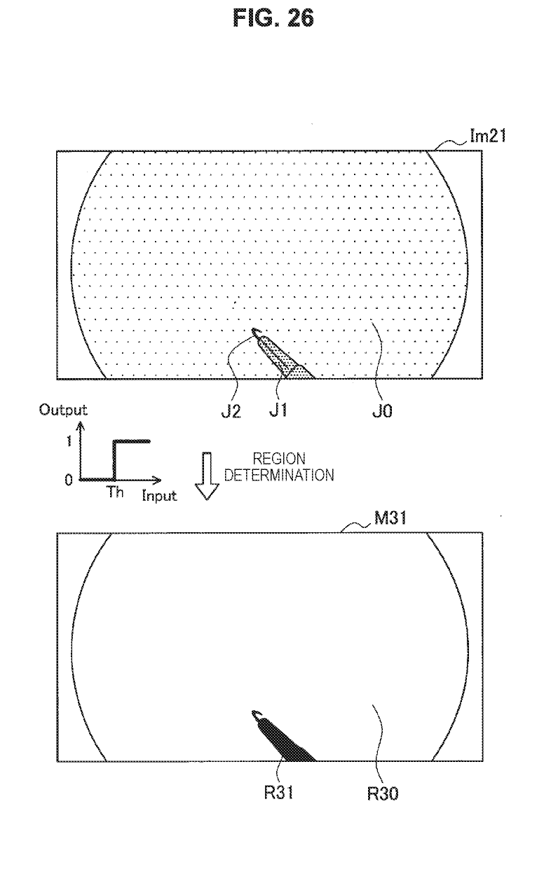

FIG. 26 is an explanatory diagram for describing still another example of a result of determination of a non-living body region using a region-determination image. An upper part of FIG. 26 illustrates a region-determination image Im21 that is a far infrared image. A subject J0 appearing in the region-determination image Im21 is a living body absorbing far infrared light, and therefore a pixel value of a region corresponding to the subject J0 indicates a relatively low value. In contrast, subjects J1 and J2, appearing in the region-determination image Im21 are non-living bodies that reflect far infrared light, and therefore pixel values of regions corresponding to the subjects J1 and J2, indicate relatively high values. Results of such region determination based on comparison between the pixel values in the region-determination image Im21 and the threshold are shown by mask information M31 illustrated in a lower part of FIG. 26. The mask information M31 divides the whole image region into a living body region R30 and a non-living body region R31 (e.g., with a binary value of each pixel).

The region determination unit 350 may execute, for example, a segmentation technique such as a graph cut method, thereby segmenting the illustrated single non-living body region R31 into a plurality of non-living body regions.

### (3) Storage Unit

The storage unit 360 stores information for various types of processing executed in the CCU 51. For example, the storage unit 360 stores threshold information to be used by the region determination unit 350 for region determination.

### (4) Disparity Determination Unit

The disparity determination unit 370 determines a disparity by using the disparity-determination image input from the signal acquisition unit 340. More specifically, the disparity determination unit 370, as well as the disparity determination unit 170 according to the first embodiment and the disparity determination unit 270 according to the second embodiment, determines at least a disparity to be applied to the non-living body region by using not only the disparity-determination image but also a result of the determination of the non-living body region. In the present embodiment, the non-living body region is determined by the region determination unit 350 by using the region-determination image that is a far infrared image as described above. The disparity determination unit 370 may set a size of a collation block depending on a size or shape of the non-living body region determined by the region determination unit 350. Further, the disparity determination unit 370 may set a weight of each pixel in the collation block, which is to be used for collation, depending on the shape of the non-living body region. Typically, a weight of a pixel corresponding to the non-living body region in the collation block can be set to a first value (e.g., 1), and a weight of a pixel corresponding to the living body region in the collation block can be set to a second value (e.g., zero). The second value can mean that a pixel value at a corresponding pixel position is not added to a collation determination index or is added thereto as an extremely small value. In a case where the region determination unit 350 determines that a plurality of non-living body regions exist in the visual field, the disparity determination unit 370 may individually set a collation block in each non-living body region and determine a disparity to be applied to each non-living body region by using the set collation block. Then, the disparity determination unit 370 determines a disparity at each pixel position (or pixel block position) by using the collation block adaptively set in the non-living body region and by using a predetermined collation block in the living body region. Then, the disparity determination unit 370 outputs disparity information indicating a result of the determination to the image processing unit 180. Further, the disparity determination unit 370 outputs the disparity-determination image to the image processing unit 180. The disparity information generated by the disparity determination unit 370 may be used in the image processing unit 180 for various purposes such as generation of a stereoscopic image and extension of a depth of field as in the first embodiment and the second embodiment.

### [4-3. Flow of Processing]

In this section, examples of a flow of processing that can be executed by the camera head 13 and the CCU 51 in the above-mentioned embodiment will be described with reference to several flowcharts.

### (1) Whole Flow

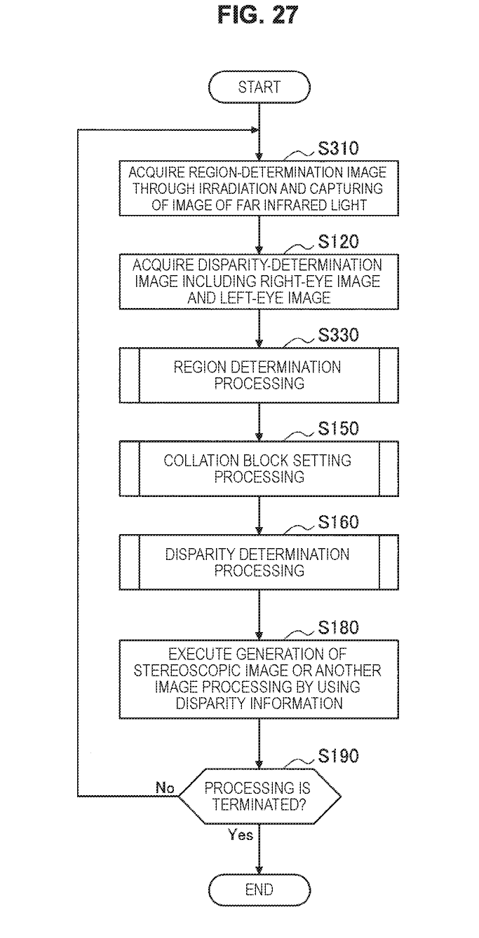

FIG. 27 is a flowchart showing an example of the whole flow of image processing according to the third embodiment. When referring to FIG. 27, first, the signal acquisition unit 340 acquires an image signal of a region-determination image through emitting the far infrared light from the second irradiation unit 315 and capturing the image in the second imaging unit 325 of the camera head 13 as an image of far infrared light (Step S310). Further, the signal acquisition unit 340 acquires an image signal of a disparity-determination image including a right-eye image and a left-eye image (Step S120).

Then, the region determination unit 350 executes region determination processing for determining a non-living body region in an observed visual field by using the region-determination image supplied from the signal acquisition unit 340 (Step S330). An example of a more detailed flow of the region determination processing executed herein will be further described below.

Then, the disparity determination unit 370 executes collation block setting processing on the basis of a result of the region determination processing in Step S330 and sets a collation block to be used for determination of a disparity of the non-living body region in the observed visual field (Step S150). The collation block setting processing executed herein may be similar to the processing described in the first embodiment.

Then, the disparity determination unit 370 executes disparity determination processing by using the collation block that has been set in the collation block setting processing in Step S150 and determines at least a disparity to be applied to the non-living body region of the disparity-determination image (Step S160). The disparity determination processing executed herein may be similar to the processing described in the first embodiment.

Then, the image processing unit 180 executes generation of a stereoscopic image or another image processing by using disparity information generated as a result of the disparity determination processing in Step S160 (Step S180).

Steps S310 to S180 described above are repeated until a termination condition of the image processing is satisfied (Step S190). For example, when user input to give an instruction to terminate the processing is detected via the input device 59, the above processing is terminated.

### (2) Region Determination Processing

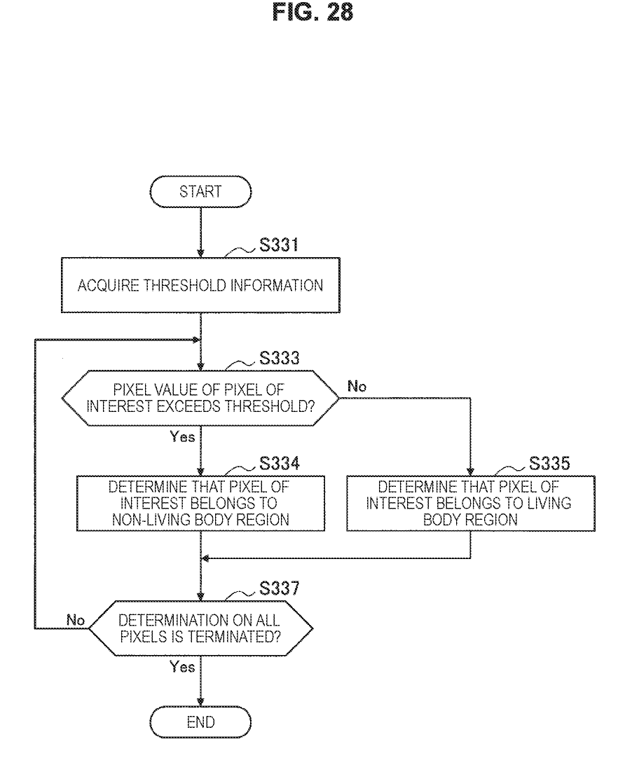

FIG. 28 is a flowchart showing an example of the detailed flow of the region determination processing shown in FIG. 27. When referring to FIG. 28, first, the region determination unit 350 acquires threshold information that can be defined in advance from the storage unit 360 (Step S331).

Then, the region determination unit 350 determines whether or not a pixel value of a pixel of interest exceeds a threshold indicated by the threshold information acquired in Step S331 (Step S333). In a case where the pixel value of the pixel of interest exceeds the threshold, the region determination unit 350 determines that the pixel of interest belongs to the non-living body region (Step S334). In contrast, in a case where the pixel value of the pixel of interest does not exceed the threshold, the region determination unit 350 determines that the pixel of interest belongs to the living body region (Step S335).

The region determination unit 350 repeats Steps S333 to S335 described above until determination on all pixels is terminated (Step S337). Then, when mask information indicating the region determination result of all the pixels is generated, the region determination processing shown in FIG. 28 is terminated.

### 5. Conclusion

Hereinabove, the embodiments of the technology of the present disclosure have been described in detail with reference to FIGS. 1 to 28. According to the above-mentioned embodiments, a non-living body region in a visual field is determined by using a region-determination image showing the visual field that is the same as a visual field that a disparity-determination image shows, and at least a disparity to be applied to the non-living body region is determined by using a result of the determination of the non-living body region and the disparity-determination image. Therefore, in a medical scene such as surgical operation or diagnosis, it is possible to improve accuracy of determination of a disparity of a non-living body subject that appears as a relatively small subject, as compared to subjects therearound, and achieve improved visibility.

Further, according to the above-mentioned embodiments, the disparity is determined by collating two disparity-determination images by using a collation block that is set on the basis of the result of the determination of the non-living body region. That is, it is possible to adaptively change setting of a collation block in accordance with the result of the determination of the non-living body region. For example, an influence of a living body region around the non-living body region on determination of a disparity is reduced by adaptively setting a collation block, and therefore accuracy of determination of the disparity to be applied to the non-living body region is effectively improved.

Further, according to the above-mentioned embodiments, in a case where a plurality of non-living bodies exist in the visual field, a collation block can be individually set in each non-living body region in the disparity-determination image. In this case, even in a case where the individual non-living bodies do not have a uniform depth, it is possible to accurately determine a disparity of each non-living body.

According to a certain embodiment, the non-living body region is determined on the basis of an already-known spectral characteristic of a subject. In this case, it is possible to determine the non-living body region by using a general image sensor capable of capturing an image of light of a visible light region. Further, it is possible to improve accuracy of determination of disparities of various non-living body subjects only by grasping expected spectral characteristics of subjects in advance. This is particularly advantageous in a medical scene in which the expected types of subjects are limited, as compared to a scene of daily life.

According to another embodiment, the non-living body region is determined on the basis of a pixel value of a region-determination image that is a far infrared image. In this case, it is possible to easily determine the non-living body region, without requiring spectral characteristic information. A method of generating a region-determination image by capturing an image of far infrared light radiating from a living body does not need irradiation of far infrared light. Therefore, it is possible to maintain a small configuration of the camera head, and no amount of heat is applied to the subject. A method of generating a region-determination image by capturing an image of reflected light of far infrared light emitted toward the subject restrains an amount of noise appearing in the far infrared image and improves accuracy of region determination.

Note that examples of the image processing system including a surgical endoscope have been mainly described in the present specification. However, the technology according to the present disclosure is not limited to such examples and is also applicable to other types of medical observation devices such as a microscope. Further, the technology according to the present disclosure may be achieved as an image processing module (e.g., image processing chip) or camera module to be mounted on such medical observation devices.

The image processing described in the present specification may be achieved by using any one of software, hardware, and a combination of software and hardware. Programs forming software are stored in advance on, for example, a storage medium (non-transitory medium) provided inside or outside each device. In addition, each program is read into a random access memory (RAM) at the time of, for example, execution and is executed by a processor such as a CPU.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

A medical image processing device including:

a region determination unit configured to determine a non-living body region in a visual field by using a region-determination image showing the visual field that is same as a visual field that a disparity-determination image shows; and

a disparity determination unit configured to determine at least a disparity to be applied to the non-living body region by using a result of the determination of the non-living body region performed by the region determination unit and the disparity-determination image.

(2)

The medical image processing device according to (1 ), in which

the disparity determination unit determines the disparity by collating a first disparity-determination image with a second disparity-determination image by using a collation block that is set on a basis of the result of the determination of the non-living body region.

(3)

The medical image processing device according to (2), in which

the disparity determination unit sets a size of the collation block depending on a size or shape of the non-living body region.

(4)

The medical image processing device according to (2) or (3), in which

the disparity determination unit sets a weight of each pixel in the collation block depending on a shape of the non-living body region, the weight being used for the collation.

(5)

The medical image processing device according to (4), in which

the disparity determination unit sets the weight of a pixel corresponding to the non-living body region in the collation block to a first value and sets the weight of a pixel corresponding to a living body region in the collation block to a second value, and

the second value means that a pixel value at a corresponding pixel position is not added to an index for the collation or is added to the index as an extremely small value.

(6)

The medical image processing device according to (4), in which

the region determination unit calculates a probability that each pixel in the region-determination image belongs to the non-living body region, and

the disparity determination unit sets the weight of each pixel in the collation block depending on a probability that the pixel belongs to the non-living body region.

(7)

The medical image processing device according to any one of (2) to (6), in which

the region determination unit segments the region-determination image into a living body region and one or more non-living body regions, and

in a case where the region determination unit determines that a plurality of the non-living body regions exist, the disparity determination unit individually sets the collation block in each of the non-living body regions and determines a disparity to be applied to each of the non-living body regions by using the set collation block.

(8)

The medical image processing device according to any one of (1) to (7), in which

the region-determination image is generated by capturing an image of reflected light of light emitted toward a subject in the visual field, the light having a wavelength whose spectral characteristic of at least one of a living body and a non-living body is already known, and

the region determination unit determines the non-living body region in the visual field by analyzing the spectral characteristic of the subject on a basis of the region-determination image.

(9)

The medical image processing device according to (8), in which

the region-determination image is generated by capturing an image of reflected light of light emitted toward the subject in the visual field a plurality of times in different wavelength patterns.

(10)

The medical image processing device according to (8) or (9), further including

a storage unit configured to store spectral characteristic information indicating the spectral characteristic of the at least one of the living body and the non-living body.

(11)

The medical image processing device according to any one of (1) to (7), in which

the region-determination image is a far infrared image.

(12)

The medical image processing device according to (11), in which

the region-determination image is generated by capturing an image of far infrared light radiating from a subject in the visual field, and

the region determination unit determines that a pixel indicating a pixel value below a threshold in the region-determination image belongs to the non-living body region.

(13)

The medical image processing device according to (11), in which

the region-determination image is generated by capturing an image of reflected light of far infrared light emitted toward a subject in the visual field, and

the region determination unit determines that a pixel indicating a pixel value exceeding a threshold in the region-determination image belongs to the non-living body region.

(14)

The medical image processing device according to any one of (1) to (13), further including

an image processing unit configured to generate a stereoscopic image corresponding to the visual field on a basis of the disparity determined by the disparity determination unit.

(15)

The medical image processing device according to any one of (1) to (14), further including

an image processing unit configured to execute extended depth of field processing on a basis of the disparity determined by the disparity determination unit.

(16)

The medical image processing device according to any one of (1) to (15), further including

an image processing unit configured to emphasize a stereoscopic effect of a displayed stereoscopic image by correcting the disparity determined by the disparity determination unit.

(17)

A medical image processing system including:

the medical image processing device according to any one of (1) to (15); and

an imaging device configured to capture an image of a subject in the visual field and generate at least one of the disparity-determination image and the region-determination image.

(18)

A medical image processing method including:

determining a non-living body region in a visual field by using a region-determination image showing the visual field that is same as a visual field that a disparity-determination image shows; and

determining at least a disparity to be applied to the non-living body region by using a result of the determination of the non-living body region and the disparity-determination image.

(19)

A program for causing a processor that controls a medical image processing device to function as:

a region determination unit configured to determine a non-living body region in a visual field by using a region-determination image showing the visual field that is same as a visual field that a disparity-determination image shows; and

a disparity determination unit configured to determine at least a disparity to be applied to the non-living body region by using a result of the determination of the non-living body region performed by the region determination unit and the disparity-determination image.

### REFERENCE SIGNS LIST

1 medical image processing system
13 camera head (imaging device)
51 CCU (image processing device)
140, 240, 340 signal acquisition unit
150, 250, 350 region determination unit
160, 260, 360 storage unit
170, 270, 370 disparity determination unit
180 image processing unit
190 control unit

1. A medical image processing device comprising:
a region determination unit configured to determine a non-living body region in a visual field by using a region-determination image showing the visual field that is same as a visual field that a disparity-determination image shows; and
a disparity determination unit configured to determine at least a disparity to be applied to the non-living body region by using a result of the determination of the non-living body region performed by the region determination unit and the disparity-determination image.

2. The medical image processing device according to claim 1, wherein
the disparity determination unit determines the disparity by collating a first disparity-determination image with a second disparity-determination image by using a collation block that is set on a basis of the result of the determination of the non-living body region.

3. The medical image processing device according to claim 2, wherein
the disparity determination unit sets a size of the collation block depending on a size or shape of the non-living body region.

4. The medical image processing device according to claim 2, wherein
the disparity determination unit sets a weight of each pixel in the collation block depending on a shape of the non-living body region, the weight being used for the collation.

5. The medical image processing device according to claim 4, wherein
the disparity determination unit sets the weight of a pixel corresponding to the non-living body region in the collation block to a first value and sets the weight of a pixel corresponding to a living body region in the collation block to a second value, and
the second value means that a pixel value at a corresponding pixel position is not added to an index for the collation or is added to the index as an extremely small value.

6. The medical image processing device according to claim 4, wherein
the region determination unit calculates a probability that each pixel in the region-determination image belongs to the non-living body region, and
the disparity determination unit sets the weight of each pixel in the collation block depending on a probability that the pixel belongs to the non-living body region.

7. The medical image processing device according to claim 2, wherein
the region determination unit segments the region-determination image into a living body region and one or more non-living body regions, and
in a case where the region determination unit determines that a plurality of the non-living body regions exist, the disparity determination unit individually sets the collation block in each of the non-living body regions and determines a disparity to be applied to each of the non-living body regions by using the set collation block.

8. The medical image processing device according to claim 1, wherein
the region-determination image is generated by capturing an image of reflected light of light emitted toward a subject in the visual field, the light having a wavelength whose spectral characteristic of at least one of a living body and a non-living body is already known, and
the region determination unit determines the non-living body region in the visual field by analyzing the spectral characteristic of the subject on a basis of the region-determination image.

9. The medical image processing device according to claim 8, wherein
the region-determination image is generated by capturing an image of reflected light of light emitted toward the subject in the visual field a plurality of times in different wavelength patterns.

10. The medical image processing device according to claim 8, further comprising
a storage unit configured to store spectral characteristic information indicating the spectral characteristic of the at least one of the living body and the non-living body.

11. The medical image processing device according to claim 1, wherein
the region-determination image is a far infrared image.

12. The medical image processing device according to claim 11, wherein
the region-determination image is generated by capturing an image of far infrared light radiating from a subject in the visual field, and
the region determination unit determines that a pixel indicating a pixel value below a threshold in the region-determination image belongs to the non-living body region.

13. The medical image processing device according to claim 11, wherein
the region-determination image is generated by capturing an image of reflected light of far infrared light emitted toward a subject in the visual field, and
the region determination unit determines that a pixel indicating a pixel value exceeding a threshold in the region-determination image belongs to the non-living body region.

14. The medical image processing device according to claim 1, further comprising
an image processing unit configured to generate a stereoscopic image corresponding to the visual field on a basis of the disparity determined by the disparity determination unit.

15. The medical image processing device according to claim 1, further comprising
an image processing unit configured to execute extended depth of field processing on a basis of the disparity determined by the disparity determination unit.

16. The medical image processing device according to claim 1, further comprising
an image processing unit configured to emphasize a stereoscopic effect of a displayed stereoscopic image by correcting the disparity determined by the disparity determination unit.

17. A medical image processing system comprising:
the medical image processing device according to claim 1; and
an imaging device configured to capture an image of a subject in the visual field and generate at least one of the disparity-determination image and the region-determination image.

18. A medical image processing method comprising:
determining a non-living body region in a visual field by using a region-determination image showing the visual field that is same as a visual field that a disparity-determination image shows; and
determining at least a disparity to be applied to the non-living body region by using a result of the determination of the non-living body region and the disparity-determination image.

19. A program for causing a processor that controls a medical image processing device to function as:
a region determination unit configured to determine a non-living body region in a visual field by using a region-determination image showing the visual field that is same as a visual field that a disparity-determination image shows; and
a disparity determination unit configured to determine at least a disparity to be applied to the non-living body region by using a result of the determination of the non-living body region performed by the region determination unit and the disparity-determination image.
